(19) United States
(12) Patent Application Publication
(10) Pub. No.: US 2015/0143578 A1
(43) Pub. Date: May 21, 2015

(54) PLANT REGULATORY GENES PROMOTING ASSOCIATION WITH NITROGEN FIXING BACTERIA

(71) Applicant: Pontificia Universidad Católica de Chile, Santiago (CL)

(72) Inventors: Tatiana Kraiser, Santiago (CL); Bernardo González, Santiago (CL); Rodrigo A. Gutiérrez, Santiago (CL)

(21) Appl. No.: 14/083,193

(22) Filed: Nov. 18, 2013

(51) Int. Cl. C12N 15/82

(52) U.S. Cl. CPC C12N 15/8261
USPC 800/278; 800/298; 435/419; 536/23.6

(57) ABSTRACT

This disclosure concerns plant nitrogen responses. Embodiments concern regulatory factors that contribute to the functional association of plants (e.g., non-nodulating plants) with nitrogen-fixing bacteria.

### FIELD OF THE DISCLOSURE

The present disclosure relates to plant biochemistry. Embodiments relate to genetic factors regulating the association of plants with nitrogen-fixing bacteria.

### BACKGROUND

Nitrogen is an essential macronutrient for plant growth and development, and it is a major limiting factor with regard to plant growth and crop productivity. Marschner (1995) Mineral Nutrition of Higher Plants, Academic Press, Harcourt, San Diego, Calif., p. 889; Epstein (2005) Mineral Nutrition of Plants: Principles and Perspectives, 2^{nd} Ed., Sinauer Associates, Inc., Sunderland, Mass.; Galloway and Cowling (2002) AMBIO 31:64. Traditional agriculture is based on nitrogen fertilizers to support world nutritional needs. However, such fertilizers are damaging to the environment and to human health when used in excess.

Despite its abundance in the atmosphere, nitrogen reserves in the biosphere are not directly accessible by plants. Plants acquire nitrogen directly from the soil and in some cases can be provided by nitrogen-fixing bacteria (NFB) through the process of biological nitrogen fixation; the main process transforming atmospheric N₂ into biologically useful nitrogen forms such as ammonium in natural ecosystems (See, e.g., Olivares et al. (2013) Mol. Plant Microbe Interact. 26:486). Since nitrogen is often limiting in soils, some plant species have evolved molecular mechanisms to associate with nitrogen fixing bacteria (NFB). Zehr et al. (2003) Env. Microbiol. 5:539; Sprent and James (2007) Plant Physiol. 144:575; Kraiser et al. (2011) J. Exp. Bot. 62:1455. Bacteria and archaea are the only types of organisms capable of biological nitrogen fixation. Reduction of atmospheric N₂ to ammonium is catalyzed by the nitrogenase complex, composed of dinitrogenase and dinitrogenase reductase subunits. Joerger et al. (1991) J. Bacteriol. 173:4440.

Plant-bacteria interactions associated with nitrogen nutrition have been primarily studied in legumes. Legumes are able to symbiotically associate with a phylogenetically diverse group of bacteria, collectively called rhizobia. Kistner and Pamiske (2002) Trends Plant Sci. 7:511. Association of these plant species with their bacterial partners induces formation of a specialized organ called a “nodule.” Nodules harbor bacteria and provide appropriate conditions for nitrogen fixation to occur. Markmann and Pamiske (2009) Trends Plant Sci. 14:77. In these nodules, the plant provides carbon sources in exchange for nitrogen fixed by the bacteria. Kistner and Parniske (2002), supra; Masson-Boivin et al. (2009) Trends Mirobiol. 17:458. The symbiotic association between bacteria and legumes is highly-regulated, and it occurs only when plants are grown under nitrogen-limiting conditions. Low levels of nitrate and ammonium stimulate nodule formation, and high levels of these nutrients also inhibit the number of infection sites in the root and N-fixation in pre-existing nodules. Eaglesham (1989) Crop Sci. 29:115; Zahran (1999) Microbiol. Mol. Biol. Rev. 63:968; Bisseling et al. (1978) Bio. Biophys. Acta 539:1.

Plants recognize nodulation (Nod) factors secreted by bacteria, which activate a signal transduction pathway that includes primary transcription factors NODULATION SIGNALING PATHWAY 1 (NSP1) and NSP2. Smit et al. (2005) Science 308:1789; Kalo et al. (2005) Science 308:1786; Heckmann et al. (2006) Plant Physiol. 142:1739. Together, these transcription factors regulate the expression of NODULE INCEPTION (NIN) genes, which are required for bacteria infection and nodule organogenesis. Smit et al. (2005), supra; Hirsch et al. (2009) Plant Cell 21:545; Schauser et al. (1999) Nature 402:191. Activation of these transcription factors in response to bacterial Nod factors occurs when plants are under N-limiting conditions. Barbulova et al. (2007) Mol. Plant Microbe Interact. 20:994.

Besides legumes, a limited number of other plant species from different taxa have been reported to associate with NFB. Despite being unable to nodulate, under N-limiting conditions, some plants (e.g., wheat and sugarcane) can assimilate a significant part of their nitrogen requirements through biological N-fixation. Iniguez et al. (2004) Mol. Plant Microbe Interact. 17:1078; Boddey et al. (1991) Plant Soil 137:111.

### BRIEF SUMMARY OF THE DISCLOSURE

Classes of genes (e.g., AtNSP1-like, AtNLP4, and AtNLP9) that regulate a functional interaction for N-nutrition between non-nodulating plant species and NFB (e.g., Sinorhizobium meliloti) are disclosed.

In particular embodiments are described non-natural nucleic acid molecules comprising a polynucleotide operably linked to a heterologous promoter, wherein the polynucleotide is a polynucleotide that is at least 80% identical to SEQ ID NO:4; a polynucleotide that hybridizes under stringent (e.g., highly stringent) conditions to a nucleic acid consisting of SEQ ID NO:4; a polynucleotide that is at least 80% identical to SEQ ID NO:5; a polynucleotide that hybridizes under stringent (e.g., highly stringent) conditions to a nucleic acid consisting of SEQ ID NO:5; a polynucleotide that is at least 80% identical to SEQ ID NO:6; or a polynucleotide that hybridizes under stringent (e.g., highly stringent) conditions to a nucleic acid consisting of SEQ ID NO:6.

Also described herein are methods for increasing nitrogen efficiency in a plant (e.g., a non-nodulating plant). In some embodiments, the method may comprise introducing at least one heterologous polypeptide into the plant to produce a transgenic plant, wherein the heterologous polypeptide is a Nodulation Signaling Pathway-like (NSP) or NIN-like Protein (NLP). In particular embodiments, the heterologous polypeptide is NSP1, NLP4, or NLP9. Such a heterologous polypeptide may be, for example, at least about 50%, about 55%, about 60%, about 65%, about 70%, about 75%, about 80%, 81%, 82%, 83%, 84%, 85%, 86%, 87%, 88%, 89%, 90%, 91%, 92%, 93%, 94%, 95% 96%, 97%, 98%, 99%, or 100% identical to one or more of SEQ ID NOs:1, 3, and 4.

In some embodiments, the method for increasing nitrogen efficiency in a plant may comprise transforming the plant with a polynucleotide encoding the heterologous polypeptide. In particular embodiments, the polynucleotide may be substantially identical to one of SEQ ID NOs:4-6, and/or be a homolog or ortholog thereof. In particular embodiments, the polynucleotide encoding the heterologous polypeptide hybridizes under stringent (e.g., highly stringent) conditions to at least one of SEQ ID NOs:4-6.

Also described herein are methods for increasing nitrogen efficiency in a plant (e.g., a non-nodulating plant) comprising introducing at least one at least one means for promoting association with NFB into the plant, for example, to produce a transgenic plant. Examples of means for promoting association with NFB include a polypeptide consisting of SEQ ID NO:1; a polypeptide consisting of SEQ ID NO:2; a polypeptide consisting of SEQ ID NO:3; a polynucleotide consisting of SEQ ID NO:4; a polynucleotide consisting of SEQ ID NO:5; and a polynucleotide consisting of SEQ ID NO:6.

Further described herein are plant cell, plant parts, plant materials, plant tissues, plant seeds, and whole plants comprising or stably transformed with any of the foregoing polypeptides and/or nucleic acid constructs. Such a transgenic plant in particular embodiments exhibits increased growth under limited nitrogen growing conditions, as compared to a wild-type plant of the same species.

The foregoing and other features will become more apparent from the following detailed description of several embodiments, which proceeds with reference to the accompanying figures.

### BRIEF DESCRIPTION OF THE FIGURES

FIG. 1(A-B) includes a representation of the effect of NFB on plant growth under limiting N conditions. Plant biomass (dry weight; n=15) was measured seven days after transfer from complete MS medium to MS medium with N (40 mM NO₃+20 mM NH₄) or without N, and inoculated or not with different bacteria (NFB: Burkholderia vietnamensis G4, B. xenovorans LB400, Cupriavidus taiwanensis LMG19424, Rhizobium etli CFN42, Sinorhizobium meliloti RMP110; and non-NFB: B. phytofirmans PSJN, C. pinatubonensis JMP134) (in order from left to right). FIG. 1(A). Plant dry weight was measured for plants grown in N-containing medium for seven days and that were transplanted to limiting N conditions and inoculated with live or dead S. meliloti, or non-inoculated. FIG. 1(B). The number of lateral roots per plant were measured for plants grown and treated in the same conditions that are detailed above for FIG. 1(A). FIG. 1(C) (mean of three independent biological replicates±SE; asterisk indicates means that differ significantly as compared to non-inoculated plants grown without N (P<0.05)).

FIG. 2(A-B) includes a representation of the enhancement of plant growth by N fixation under N-limiting conditions. Biomass was measured as dry weight of plants grown under sufficient (2.5 mM NH₄NO₃) or limiting N conditions, inoculated or not with S. meliloti RMP110 wild-type or with the mutant type unable to fix N. FIG. 2(A). Biological nitrogen fixation was measured by the ¹⁵N dilution technique. Plants were grown in a medium with sufficient N (5% ¹⁵N). After seven days, plants were transferred to plates with different treatments. Using mass spectrometry, the amounts of ¹⁴N and ¹⁵N were determined in harvested and dried plants 7 days after treatment. δ¹⁵N represents the ¹⁴N:¹⁵N isotope ratio, relative to the non-inoculated condition (mean of three independent biological replicates±SE (P<0.05)). FIG. 2(A).

FIG. 3(A-B) includes a representation of the induction of certain Arabidopsis genes upon bacterial inoculation. NSP1 (FIG. 3(A)) and NIN-like transcription factors (FIG. 3(A)) gene expression was measured by real-time quantitative reverse transcription PCR at the third or seventh day post treatment. Plants were grown under sufficient (2.5 mM NH₄NO₃) or limiting N conditions, and inoculated or not with NFB. Values plotted correspond to the mean of three independent biological replicates±SE (asterisk indicates means that differ significantly as compared to non-inoculated plants (P<0.05)).

FIG. 4 includes a representation of the effect of mutations in Arabidopsis thaliana of genes essential for a functional association with S. meliloti RMP110; AtNSP1-like, AtNLP4, AtNLP8, and AtNLP9 transcription factors. Biomass is expressed as dry weight measured in wild-type and mutant plants grown under limiting nitrogen conditions and inoculated or not with S. meliloti (mean of three independent biological replicates±SE; asterisk indicates means that differ significantly as compared to non-inoculated plants (P<0.05)).

FIG. 5 includes an image showing the effect of NFB on root hair length. Plants grown in complete MS salt medium for seven days were transplanted to MS medium without N, and inoculated or not with NFB. Pictures were taken seven days after treatment.

FIG. 6(A-B) includes a representation of the effect of S. meliloti on Arabidopsis root system architecture. Lateral root density (FIG. 6(A)) and primary root length (FIG. 6(B)) were measured in plants grown under sufficient/limiting N conditions, inoculated or not with S. meliloti RMP110 wild-type or with the mutant type unable to fix N. Values plotted correspond to the mean of three independent biological replicates±SE.

FIG. 7(A-B) includes a representation of the response to bacterium inoculation of AtNSP2-like, AtNLP1, AtNLP2, and AtNLP5. Gene expression was measured by real-time quantitative reverse transcription PCR at the third or seventh day post treatment (mean of three independent biological replicates±SE; asterisk indicates means that differ significantly as compared to non-inoculated plants (P<0.05)). Plants were grown under sufficient or limiting N conditions, and inoculated or not with S. meliloti.

### SEQUENCE LISTING

The nucleic acid sequences listed in the accompanying sequence listing are shown using standard letter abbreviations for nucleotide bases, as defined in 37 C.F.R. §1.822. Only one strand of each nucleic acid sequence is shown, but the complementary strand is understood to be included by any reference to the displayed strand. In the accompanying sequence listing:

SEQ ID NO: l shows an NSP1-like (SCL29; At3g13840)
polypeptide:
MLLEETEPPNQTLDHVLSWLEDSVSLSPLPGFDDSYLLHEFDGSQTWEWDQTQDPE
HGFIQSYSQDLSAAYVGCEATNLEVVTEAPSIDLDLPPEIQQPNDQSRKRSHDGFL
EAQQVKKSARSKRKAIKSSEKSSKDGNKEGRWAEKLLNPCALAITASNSSRVQHYL
CVLSELASSSGDANRRLAAFGLRALQHHLSSSSVSSSFWPVFTFASAEVKMFQKTL
LKFYEVSPWFALPNNMANSAILQILAQDPKDKKDLHIIDIGVSHGMQWPTLLEALS
CRLEGPPPRVRITVISDLTADIPFSVGPPGYNYGSQLLGFARSLKINLQISVLDKL
QLIDTSPHENLIVCAQFRLHHLKHSINDERGETLKAVRSLRPKGVVLCENNGECSS
SADFAAGFSKKLEYVWKFLDSTSSGFKEENSEERKLMEGEATKVLMNAGDMNEGKE
KWYERMREAGFFVEAFEEDAVDGAKSLLRKYDNNWEIRMEDGDTFAGLMWKGEAVS
FCSLWK
SEQ ID NO: 2 shows an NLP4 (At1g20640) polypeptide:
MEDSFLQSENVVMDADFMDGLLLDGCWLETTDGSEFLNIAPSTSSVSPFDPTSFMW
SPTQDTSALCTSGVVSQMYGQDCVERSSLDEFQWNKRWWIGPGGGGSSVTERLVQA
VEHIKDYTTARGSLIQLWVPVNRGGKRVLTTKEQPFSHDPLCQRLANYREISVNYH
FSAEQDDSKALAGLPGRVFLGKLPEWTPDVRFFKSEEYPRVHHAQDCDVRGTLAIP
VFEQGSKICLGVIEVVMTTEMVKLRPELESICRALQAVDLRSTELPIPPSLKGCDL
SYKAALPEIRNLLRCACETHKLPLAQTWVSCQQQNKSGCRHNDENYIHCVSTIDDA
CYVGDPTVREFHEACSEHHLLKGQGVAGQAFLTNGPCFSSDVSNYKKSEYPLSHHA
NMYGLHGAVAIRLRCIHTGSADFVLEFFLPKDCDDLEEQRKMLNALSTIMAHVPRS
LRTVTDKELEEESEVIEREEIVTPKIENASELHGNSPWNASLEEIQRSNNTSNPQN
LGLVFDGGDKPNDGFGLKRGFDYTMDSNVNESSTFSSGGFSMMAEKKRTKADKTIT
LDVLRQYFAGSLKDAAKNIGVCPTTLKRICRQHGIQRWPSRKIKKVGHSLQKIQRV
IDSVQGVSGPLPIGSFYANFPNLVSQSQEPSQQAKTTPPPPPPVQLAKSPVSSYSH
SSNSSQCCSSETQLNSGATTDPPSTDVGGALKKTSSEIELQSSSLDETILTLSSLE
NIPQGTNLLSSQDDDFLRIKVSYGEEKIRLRMRNSRRLRDLLWEIGKRFSIEDMSR
YDLKYLDEDNEWVLLTCDEDVEECVDVCRTTPSHTIKLLLQASSHHFPERSSATEY
SLWH
SEQ ID NO: 3 shows an NLP9 (At3g59580) polypeptide:
MENPSASRDNKGFCFPDIPVEEMDGWVKNLISEEDMFSSSSTSELMNFESFASWCN
SPSAADILFTQYGLSTSQSIIPFGGLEGSYACEKRPLDCTSVPRSLSHSLDEKMLK
ALSLFMEFSGEGILAQFWTPIKTGDQYMLSTCDQAYLLDSRLSGYREASRRFTFSA
EANQCSYPGLPGRVFISGVPEWTSNVMYYKTAEYLRMKHALDNEVRGSIAIPVLEA
SGSSCCAVLELVTCREKPNFDVEMNSVCRALQAVNLQTSTIPRRQYLSSNQKEALA
EIRDVLRAVCYAHRLPLALAWIPCSYSKGANDELVKVYGKNSKECSLLCIEETSCY
VNDMEMEGFVNACLEHYLREGQGIVGKALISNKPSFSSDVKTFDICEYPLVQHARK
FGLNAAVATKLRSTFTGDNDYILEFFLPVSMKGSSEQQLLLDSLSGTMQRLCRTLK
TVSDAESIDGTEFGSRSVEMTNLPQATVSVGSFHTTFLDTDVNSTRSTFSNISSNK
RNEMAGSQGTLQQEISGARRLEKKKSSTEKNVSLNVLQQYFSGSLKDAAKSLGVCP
TTLKRICRQHGIMRWPSRKINKVNRSLRKIQTVLDSVQGVEGGLKFDSVTGEFVAV
GPFIQEFGTQKSLSSHDEDALARSQGDMDEDVSVEPLEVKSHDGGGVKLEEDVETN
HQAGPGSLKKPWTWISKQSGLIYSDDTDIGKRSEEVNKDKEDLCVRRCLSSVALAG
DGMNTRIERGNGTVEPNHSISSSMSDSSNSSGAVLLGSSSASLEQNWNQIRTHNNS
GESGSSSTLTVKATYREDTVRFKLDPYVVGCSQLYREVAKRFKLQEGAFQLKYLDD
EEEWVMLVTDSDLHECFEILNGMRKHTVKFLVRDIPNTAMGSSAGSNGYLGTGT
SEQ ID NO: 4 shows an NSP1-like (SCL29; At3g13840)
polynucleotide coding sequence (CDS):
ATGTTGTTGGAAGAAACAGAACCACCAAACCAGACTCTAGATCATGTCCTAAGCTG
GCTTGAGGATTCTGTGTCCTTATCCCCATTACCAGGATTTGATGATTCTTATTTGC
TCCACGAGTTTGATGGGTCTCAAACGTGGGAATGGGATCAGACTCAAGATCCGGAG
CATGGTTTTATTCAAAGCTATAGTCAAGATCTTAGTGCAGCATATGTTGGTTGTGA
AGCAACTAACCTGGAAGTGGTAACAGAAGCTCCATCCATTGATTTGGATCTTCCAC
CTGAAATTCAGCAACCAAACGATCAGTCCAGGAAAAGGAGCCACGACGGGTTTCTC
GAGGCACAACAGGTGAAAAAATCGGCAAGGAGCAAGAGAAAAGCAATCAAGTCTAG
TGAGAAGAGCTCCAAAGATGGTAACAAGGAAGGGAGATGGGCAGAGAAGTTGCTTA
ACCCTTGTGCCTTGGCCATTACGGCAAGTAACTCATCAAGGGTTCAACATTACCTT
TGTGTTCTCTCTGAACTGGCCTCTTCTTCTGGTGATGCTAATCGTCGGCTTGCAGC
TTTTGGTCTTCGGGCTTTGCAACATCATCTTTCCTCATCCTCTGTGTCATCATCCT
TTTGGCCTGTTTTTACTTTTGCTTCAGCGGAAGTGAAGATGTTTCAAAAGACTCTG
CTTAAGTTCTACGAGGTAAGCCCTTGGTTTGCTTTGCCTAACAACATGGCAAACTC
AGCTATCCTGCAGATTTTAGCACAGGATCCCAAAGATAAAAAGGATCTTCATATTA
TTGATATTGGTGTTTCTCACGGTATGCAATGGCCCACTTTGTTGGAGGCTCTGAGC
TGCAGACTAGAAGGACCTCCTCCTCGTGTTCGAATAACCGTTATATCAGATCTAAC
CGCAGACATACCTTTCTCTGTTGGTCCACCAGGTTACAATTATGGTTCTCAACTCC
TAGGCTTTGCTCGGTCTCTCAAGATCAACCTTCAGATTAGTGTACTTGACAAGTTA
CAACTCATTGATACCTCACCTCACGAGAACTTAATCGTGTGTGCTCAGTTTAGGCT
GCATCACCTGAAGCATAGCATCAATGATGAGAGAGGCGAGACTTTGAAAGCAGTGA
GAAGTTTAAGGCCAAAAGGAGTGGTTCTTTGTGAGAACAATGGAGAATGCAGTAGT
AGTGCGGACTTTGCAGCAGGATTCTCGAAGAAACTGGAGTATGTATGGAAGTTTCT
GGATTCAACAAGCTCGGGATTTAAAGAAGAGAATAGCGAAGAGAGAAAACTAATGG
AAGGAGAGGCAACAAAGGTGTTGATGAATGCAGGAGATATGAATGAAGGAAAAGAG
AAATGGTATGAGAGGATGAGAGAAGCTGGTTTTTTTGTAGAAGCATTTGAAGAAGA
TGCAGTTGATGGAGCCAAATCCTTACTAAGAAAGTATGACAACAATTGGGAAATAA
GAATGGAAGATGGAGATACCTTTGCTGGATTAATGTGGAAAGGAGAGGCAGTTTCC
TTTTGTTCATTGTGGAAGTAG
SEQ ID NO: 5 shows an NLP4 (At1g20640) polynucleotide
coding sequence (CDS):
ATGGAAGATAGTTTCCTTCAATCTGAGAACGTGGTTATGGACGCTGACTTCATGGA
TGGATTGTTACTAGATGGTTGTTGGTTAGAGACTACAGATGGATCTGAGTTTCTTA
ACATAGCTCCTTCAACTTCTTCTGTTAGCCCTTTTGATCCAACTTCCTTCATGTGG
TCTCCAACTCAAGATACATCAGCTCTTTGCACATCAGGAGTTGTATCTCAGATGTA
TGGTCAGGATTGTGTAGAAAGATCTAGTCTTGATGAGTTTCAATGGAACAAACGAT
GGTGGATTGGACCAGGAGGTGGTGGTTCTTCGGTTACTGAGAGGTTGGTTCAAGCA
GTTGAACACATTAAAGATTACACAACAGCGAGAGGCTCACTTATTCAGTTATGGGT
TCCGGTTAATAGAGGCGGTAAGCGAGTTTTGACCACAAAGGAACAACCTTTTAGCC
ATGATCCGTTGTGTCAAAGACTTGCAAACTATAGAGAGATCTCTGTGAATTATCAC
TTCTCTGCTGAGCAAGATGATTCCAAGGCTTTAGCTGGTTTGCCTGGGAGGGTTTT
CTTGGGGAAGCTTCCTGAATGGACTCCTGATGTTAGGTTTTTCAAGAGCGAGGAGT
ATCCGAGAGTACACCATGCTCAGGACTGCGATGTCCGTGGAACGCTGGCGATTCCG
GTGTTTGAACAAGGTAGTAAGATTTGCTTGGGTGTTATTGAGGTTGTAATGACCAC
TGAGATGGTTAAACTAAGACCTGAGCTTGAAAGCATTTGCAGAGCACTTCAGGCAG
TTGATCTTAGGAGCACCGAGCTTCCGATTCCACCTTCTCTAAAGGGATGTGACTTA
TCCTACAAAGCTGCCTTACCTGAAATCCGAAACCTCTTGAGATGTGCTTGTGAGAC
TCATAAACTACCTTTAGCTCAGACATGGGTTTCTTGTCAACAGCAAAACAAAAGCG
GGTGCCGTCACAACGATGAGAACTACATCCATTGCGTATCAACCATTGATGATGCT
TGCTACGTTGGTGATCCAACAGTTCGTGAGTTCCATGAAGCTTGCTCTGAGCATCA
CCTCTTGAAAGGCCAAGGAGTTGCAGGTCAAGCCTTCTTGACCAATGGACCTTGCT
TTTCATCTGATGTATCTAACTACAAGAAATCAGAGTACCCTCTCTCTCACCATGCT
AATATGTACGGTTTACATGGCGCGGTTGCAATTCGCCTGCGGTGCATCCACACGGG
CTCTGCTGATTTCGTCTTAGAGTTCTTTTTGCCTAAAGACTGCGATGATCTGGAGG
AACAGAGGAAAATGTTGAATGCTCTTTCAACTATTATGGCTCATGTGCCTAGAAGC
TTAAGGACTGTTACAGACAAAGAACTAGAAGAAGAGAGTGAAGTGATAGAGAGGGA
AGAGATAGTAACGCCAAAGATAGAAAACGCATCTGAACTCCACGGAAATTCCCCAT
GGAATGCCTCTCTTGAAGAAATCCAGCGGAGTAATAATACTAGTAATCCTCAGAAT
CTTGGACTGGTATTTGATGGAGGAGACAAACCAAATGATGGTTTTGGCTTAAAAAG
AGGTTTTGACTACACCATGGATTCTAATGTCAATGAGAGCAGCACTTTCTCTAGTG
GTGGTTTCAGTATGATGGCCGAGAAAAAGCGTACAAAAGCAGATAAAACCATCACT
TTGGATGTTCTTCGACAGTATTTCGCTGGGAGCTTGAAAGATGCAGCCAAGAATAT
CGGTGTTTGTCCAACGACCTTGAAGAGAATATGCAGACAGCATGGTATACAAAGAT
GGCCTTCAAGAAAGATAAAAAAAGTGGGACATTCTCTGCAGAAGATCCAACGAGTG
ATTGATTCGGTTCAAGGTGTTTCTGGTCCTCTTCCCATAGGCTCATTCTATGCAAA
TTTCCCCAATTTAGTCTCACAGTCACAAGAACCATCACAACAAGCCAAGACCACGC
CTCCTCCTCCGCCGCCAGTGCAGCTTGCAAAGTCCCCTGTATCCTCGTATAGTCAC
AGTTCAAACTCTAGCCAATGTTGCTCCAGTGAAACCCAACTAAACAGCGGTGCAAC
AACCGATCCTCCTTCAACTGATGTAGGAGGTGCATTGAAGAAGACGAGCAGCGAAA
TCGAGCTTCAAAGCTCGAGTCTTGACGAGACAATTTTGACTCTCTCCAGTTTAGAA
AACATCCCTCAAGGCACCAACTTGTTATCATCTCAAGATGATGACTTTCTGAGGAT
TAAAGTTAGCTACGGAGAAGAGAAGATCAGATTACGGATGCGGAATTCGCGCAGGT
TAAGAGATCTATTGTGGGAGATTGGGAAGCGGTTTAGCATAGAGGATATGAGCAGG
TATGATCTAAAGTACTTAGACGAAGACAATGAATGGGTTTTGTTGACTTGCGACGA
AGATGTAGAAGAGTGTGTAGATGTCTGCAGAACTACACCGAGTCATACCATTAAGC
TTTTGCTTCAGGCTTCTTCTCATCATTTCCCTGAACGTTCTTCAGCTACTGAATAC
AGTTTATGGCACTGA
SEQ ID NO: 6 shows an NLP9 (At3g59580) polynucleotide
coding sequence (CDS):
ATGGAGAACCCATCAGCATCCAGAGATAATAAAGGTTTCTGTTTTCCAGATATTCC
AGTAGAAGAAATGGATGGCTGGGTTAAGAATTTGATCTCTGAAGAAGATATGTTTA
GCTCCTCTTCAACTTCAGAGCTTATGAATTTCGAATCTTTTGCTTCATGGTGCAAC
AGCCCTTCCGCTGCAGATATCTTGTTCACTCAATACGGTTTATCGACCTCTCAATC
TATTATACCTTTCGGAGGCTTAGAAGGCTCATACGCTTGCGAGAAAAGACCGTTAG
ACTGTACTAGTGTTCCAAGGTCATTGAGCCATTCTCTTGATGAGAAGATGCTCAAA
GCATTAAGTTTGTTTATGGAGTTCTCTGGAGAGGGAATTCTGGCACAGTTTTGGAC
TCCTATTAAGACAGGAGATCAGTACATGCTTAGTACTTGTGATCAGGCGTATCTGC
TTGACTCGAGGCTATCTGGATACCGTGAAGCGTCGAGGAGATTCACTTTCTCTGCT
GAAGCAAATCAATGCTCTTATCCAGGTCTTCCAGGCAGAGTCTTTATCTCTGGAGT
TCCTGAGTGGACATCAAACGTTATGTATTACAAGACTGCTGAATATTTAAGGATGA
AGCATGCATTAGATAACGAAGTCCGTGGTTCGATTGCAATTCCTGTCCTTGAAGCA
TCAGGTTCTTCTTGTTGTGCAGTTCTGGAACTTGTGACATGTAGGGAAAAACCAAA
CTTTGATGTGGAGATGTACTCTGTTTGCCGTGCTCTGCAGGCCGTGAACTTACAAA
CATCAACTATTCCTCGTCGCCAGTACCTTTCAAGTAATCAAAAAGAAGCTTTGGCT
GAAATAAGAGATGTTCTCAGAGCAGTGTGCTATGCACATAGGTTGCCTTTAGCTCT
AGCTTGGATTCCCTGTAGTTACTCCAAAGGAGCAAACGATGAGTTGGTAAAGGTTT
ATGGAAAAAACTCAAAGGAATGTTCTCTTCTTTGCATAGAAGAGACATCATGTTAT
GTGAATGATATGGAAATGGAAGGCTTTGTGAATGCATGTTTGGAGCATTATCTAAG
AGAAGGGCAAGGAATTGTTGGCAAAGCACTCATATCAAACAAACCGTCTTTCTCAT
CTGATGTAAAGACATTTGATATCTGCGAGTACCCTCTTGTTCAACATGCTCGAAAG
TTTGGTCTTAATGCTGCAGTTGCTACCAAACTGAGGAGCACATTCACTGGTGACAA
TGACTATATACTTGAGTTTTTTTTACCTGTAAGTATGAAGGGAAGCTCAGAACAAC
AACTTTTGCTGGACAGTCTCTCGGGCACCATGCAGAGACTATGTCGGACTCTGAAA
ACTGTTTCAGATGCTGAATCAATTGACGGTACAGAATTTGGATCTCGTAGTGTAGA
AATGACAAATCTCCCACAGGCTACTGTATCCGTTGGAAGCTTTCATACGACATTTC
TTGATACTGACGTCAACTCTACTCGAAGTACCTTTTCGAACATCTCCTCTAATAAA
AGAAATGAAATGGCAGGTTCTCAAGGCACTCTTCAGCAGGAAATTAGCGGAGCAAG
AAGATTAGAGAAGAAGAAAAGCAGTACAGAGAAGAATGTGAGCTTAAATGTTCTCC
AACAATACTTCTCTGGGAGCTTAAAGGATGCTGCAAAAAGCCTTGGTGTTTGTCCG
ACTACACTAAAAAGGATATGTAGACAACACGGAATTATGAGATGGCCATCTCGAAA
GATTAACAAAGTGAATAGGTCACTAAGGAAAATACAGACGGTGCTTGACTCTGTCC
AGGGTGTAGAAGGAGGACTGAAGTTTGACTCGGTGACAGGGGAATTTGTAGCAGTT
GGCCCTTTTATACAAGAATTTGGGACCCAAAAGAGTCTGTCTTCTCATGATGAAGA
TGCACTTGCAAGAAGCCAAGGTGATATGGATGAAGATGTGTCAGTAGAGCCTTTGG
AAGTTAAATCTCATGATGGTGGCGGTGTCAAGTTGGAGGAGGATGTTGAAACAAAC
CACCAAGCGGGACCAGGATCCTTGAAGAAGCCATGGACTTGGATAAGCAAACAGTC
TGGCTTGATCTATAGTGATGATACCGACATAGGAAAAAGAAGTGAAGAGGTAAACA
AGGATAAAGAAGACCTTTGTGTTCGAAGGTGCTTGAGCTCTGTAGCACTTGCAGGT
GATGGAATGAATACAAGAATCGAGCGAGGTAATGGAACTGTAGAACCAAACCACTC
CATATCAAGTAGCATGTCGGATTCATCAAATAGCTCAGGAGCAGTTTTGCTGGGAA
GTTCATCTGCTTCCTTGGAACAAAACTGGAACCAAATAAGAACTCATAACAATAGC
GGTGAAAGCGGATCAAGTTCAACACTAACCGTAAAAGCCACTTACAGAGAGGACAC
TGTACGTTTCAAGCTTGATCCATACGTTGTTGGGTGTTCTCAGCTCTACAGAGAAG
TGGCTAAGCGTTTCAAGCTGCAAGAAGGTGCCTTTCAGTTGAAATACTTGGATGAT
GAAGAAGAATGGGTGATGTTGGTCACAGATTCTGATCTCCATGAATGCTTCGAGAT
ATTAAATGGTATGAGAAAACATACAGTGAAGTTTCTGGTCCGTGATATACCGAACA
CCGCAATGGGAAGTTCCGCAGGCAGCAATGGTTACCTCGGAACAGGCACCTAA

### DETAILED DESCRIPTION

### I. Overview of Several Embodiments

In legumes, functional association between the plant and NFB is highly regulated. Molecular regulatory systems prevent the formation of nodules in plants grown in the presence of nitrogen. Only under N-limiting conditions and in the presence of the NFB, a symbiotic signal transduction pathway is activated to induce nodule development in the plant. Upon Nod factor recognition, calcium oscillation generates the induction of the primary transcription factors, NSP1 and NSP2, which induce NIN gene expression. Some components of the symbiotic transduction pathway are shared between rhizobium and arbuscular mycorrhizal fungi associations. Oldroyd (2013), supra. However and unlike NSP2, NSP1 and NIN genes are specifically implicated in the association with NFB.

Described herein is a mechanism that promotes association of non-nodulating plant species with NFB for improved N-nutrition. Under N-limiting conditions, Arabidopsis associates with Sinorhizobium meliloti, a NFB capable of providing reduced nitrogen for plant nutrition, contributing to plant growth under N-limiting conditions. Embodiments herein employ plant transcription factors identified for the first time as essential for functional association between non-nodulating plants and S. meliloti. We have discovered that homologous genes of transcription factors that are specific for the legume:NFB association are necessary to mediate plant growth promotion induced by S. meliloti in non-nodulating plants. In embodiments, the conservation of the association mechanism across plant species allows for the association between Arabidopsis and S. meliloti to be recapitulated in other plants through genetic modification, to govern beneficial interactions with NFB for N-nutrition, for example, to enhance nitrogen use efficiency in a cropping system.

As described herein, Arabidopsis is functionally associated with S. meliloti, a bacterium that associates with the legume, Medicago sativa. Marsh et al. (2007) Plant Physiol. 144:324; Peiter et al. (2007) Plant Physiol. 145:192. Under free-living conditions and in association with legumes, S. meliloti performs N-fixation only under N-limiting conditions. Szeto et al. (1987) J. Bacteriol. 169:1423. As further described herein, S. meliloti promotes plant growth under N-limiting conditions (e.g., in a medium without an additional N-source), and is thus suitable for N-fixation and the promotion of plant growth due to BNF.

### II. Abbreviations

BNF biological nitrogen fixation

N nitrogen

NFB N-fixing bacterium

NLP NIN-like protein

Nod nodulation (factor)

NIN nodule inception (gene)

### III. Terms

In the description and tables which follow, a number of terms are used. In order to provide a clear and consistent understanding of the specification and claims, including the scope to be given such terms, the following definitions are provided:

Backcrossing: Backcrossing methods may be used to introduce a nucleic acid sequence into plants. The backcrossing technique has been widely used for decades to introduce new traits into plants. Jensen, N., Ed. Plant Breeding Methodology, John Wiley & Sons, Inc., 1988. In a typical backcross protocol, the original variety of interest (recurrent parent) is crossed to a second variety (non-recurrent parent) that carries a gene of interest to be transferred. The resulting progeny from this cross are then crossed again to the recurrent parent, and the process is repeated until a plant is obtained wherein essentially all of the desired morphological and physiological characteristics of the recurrent plant are recovered in the converted plant, in addition to the transferred gene from the non-recurrent parent.

Isolated: An “isolated” biological component (such as a nucleic acid or protein) has been substantially separated, produced apart from, or purified away from other biological components in the cell of the organism in which the component naturally occurs (i.e., other chromosomal and extra-chromosomal DNA and RNA, and proteins), while effecting a chemical or functional change in the component (e.g., a nucleic acid may be isolated from a chromosome by breaking chemical bonds connecting the nucleic acid to the remaining DNA in the chromosome). Nucleic acid molecules and proteins that have been “isolated” include nucleic acid molecules and proteins purified by standard purification methods, wherein there has been a chemical or functional change in the nucleic acid or protein. The term also embraces nucleic acids and proteins prepared by recombinant expression in a host cell, as well as chemically-synthesized nucleic acid molecules, proteins, and peptides.

Nucleic acid molecule: As used herein, the term “nucleic acid molecule” may refer to a polymeric form of nucleotides, which may include both sense and anti-sense strands of RNA, cDNA, genomic DNA, and synthetic forms and mixed polymers of the above. A nucleotide may refer to a ribonucleotide, deoxyribonucleotide, or a modified form of either type of nucleotide. A “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A nucleic acid molecule can include either or both naturally occurring and modified nucleotides linked together by naturally occurring and/or non-naturally occurring nucleotide linkages.

Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hairpinned, circular, and padlocked conformations.

Oligonucleotide: An oligonucleotide is a short nucleic acid molecule. Oligonucleotides may be formed by cleavage of longer nucleic acid segments, or by polymerizing individual nucleotide precursors. Automated synthesizers allow the synthesis of oligonucleotides up to several hundred base pairs in length. Because oligonucleotides may bind to a complementary nucleotide sequence, they may be used as probes for detecting DNA or RNA. Oligonucleotides composed of DNA (oligodeoxyribonucleotides) may be used in PCR, a technique for the amplification of small DNA sequences. In PCR, the oligonucleotide is typically referred to as a “primer,” which allows a DNA polymerase to extend the oligonucleotide and replicate the complementary strand.

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, inter-nucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hairpinned, circular, and padlocked conformations.

As used herein with respect to DNA, the term “coding sequence” refers to a nucleotide sequence that is transcribed into RNA when placed under the control of appropriate regulatory sequences. A “protein coding sequence” is a nucleotide sequence (DNA or RNA) that is ultimately translated into a polypeptide, via transcription and mRNA. With respect to RNA, the term “coding sequence” refers to a nucleotide sequence that is translated into a peptide, polypeptide, or protein. The boundaries of a coding sequence are determined by a translation start codon at the 5′-terminus and a translation stop codon at the 3′-terminus. Coding sequences include, but are not limited to: genomic DNA; cDNA; EST; and recombinant nucleotide sequences.

Genome: As used herein, the term “genome” refers to chromosomal DNA found within the nucleus of a cell, and also refers organelle DNA found within subcellular components of the cell. In some embodiments of the invention, a DNA molecule may be introduced into a plant cell such that the DNA molecule is integrated into the genome of the plant cell. In these and further embodiments, the DNA molecule may be either integrated into the nuclear DNA of the plant cell, or integrated into the DNA of the chloroplast or mitochondrion of the plant cell.

Endogenous: The term “endogenous,” as applied to nucleic acids (e.g., polynucleotides, DNA, RNA, and genes) herein, refers to one or more nucleic acid(s) that are normally (e.g., in a wild-type cell of the same type and species) present within their specific environment or context. For example, an endogenous gene is one that is normally found in the particular cell in question and in the same context (e.g., with regard to regulatory sequences). Endogenous nucleic acids can be distinguished from exogenous and/or heterologous, for example and without limitation, by detection in the latter of sequences that are consequent with recombination from bacterial plasmid; identification of atypical codon preferences; and amplification of atypical sequences in a PCR reaction from primers characterized in a wild-type cell.

Exogenous: The term “exogenous,” as applied to nucleic acids herein, refers to one or more nucleic acid(s) that are not normally present within their specific environment or context. For example, if a host cell is transformed with a nucleic acid that does not occur in the untransformed host cell in nature, then that nucleic acid is exogenous to the host cell. The term exogenous, as used herein, also refers to one or more nucleic acid(s) that are identical in sequence to a nucleic acid already present in a host cell, but that are located in a different cellular or genomic context than the nucleic acid with the same sequence already present in the host cell. For example, a nucleic acid that is integrated in the genome of the host cell in a different location than a nucleic acid with the same sequence is normally integrated in the genome of the host cell is exogenous to the host cell. Furthermore, a nucleic acid (e.g., a DNA molecule) that is present in a plasmid or vector in the host cell is exogenous to the host cell when a nucleic acid with the same sequence is only normally present in the genome of the host cell.

Heterologous: The term “heterologous,” as applied to nucleic acids (e.g., polynucleotides, DNA, RNA, and genes) herein, means of different origin. For example, if a host cell is transformed with a nucleic acid that does not occur in the untransformed host cell in nature, then that nucleic acid is heterologous (and exogenous) to the host cell. Furthermore, different elements (e.g., promoter, enhancer, coding sequence, terminator, etc) of a transforming nucleic acid may be heterologous to one another and/or to the transformed host. The term heterologous, as used herein, may also be applied to one or more nucleic acid(s) that are identical in sequence to a nucleic acid already present in a host cell, but that are now linked to different additional sequences and/or are present at a different copy number, etc.

Sequence identity: The term “sequence identity” or “identity,” as used herein in the context of two nucleic acid or polypeptide sequences, may refer to the residues in the two sequences that are the same when aligned for maximum correspondence over a specified comparison window.

As used herein, the term “percentage of sequence identity” may refer to the value determined by comparing two optimally aligned sequences (e.g., nucleic acid sequences, and amino acid sequences) over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity.

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, Md.), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method.

Specifically hybridizable/specifically complementary: As used herein, the terms “specifically hybridizable” and “specifically complementary” are terms that indicate a sufficient degree of complementarity such that stable and specific binding occurs between the nucleic acid molecule and a target nucleic acid molecule. Hybridization between two nucleic acid molecules involves the formation of an anti-parallel alignment between the nucleic acid sequences of the two nucleic acid molecules. The two molecules are then able to form hydrogen bonds with corresponding bases on the opposite strand to form a duplex molecule that, if it is sufficiently stable, is detectable using methods well known in the art. A nucleic acid molecule need not be 100% complementary to its target sequence to be specifically hybridizable. However, the amount of sequence complementarity that must exist for hybridization to be specific is a function of the hybridization conditions used.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Calculations regarding hybridization conditions required for attaining particular degrees of stringency are known to those of ordinary skill in the art, and are discussed, for example, in Sambrook et al. (ed.) Molecular Cloning: A Laboratory Manual, 2^{nd} ed., vol. 1-3, Cold Spring Harbor Laboratory Press, Cold Spring Harbor, N.Y., 1989, chapters 9 and 11; and Hames and Higgins (eds.) Nucleic Acid Hybridization, IRL Press, Oxford, 1985. Further detailed instruction and guidance with regard to the hybridization of nucleic acids may be found, for example, in Tijssen, “Overview of principles of hybridization and the strategy of nucleic acid probe assays,” in Laboratory Techniques in Biochemistry and Molecular Biology-Hybridization with Nucleic Acid Probes, Part I, Chapter 2, Elsevier, N.Y., 1993; and Ausubel et al., Eds., Current Protocols in Molecular Biology, Chapter 2, Greene Publishing and Wiley-Interscience, NY, 1995.

As used herein, “stringent conditions” encompass conditions under which hybridization will only occur if there is less than 20% mismatch between the hybridization molecule and a homologous sequence within the target nucleic acid molecule. “Stringent conditions” include further particular levels of stringency. Thus, as used herein, “moderate stringency” conditions are those under which molecules with more than 20% sequence mismatch will not hybridize; conditions of “high stringency” are those under which sequences with more than 10% mismatch will not hybridize; and conditions of “very high stringency” are those under which sequences with more than 5% mismatch will not hybridize.

The following are representative, non-limiting hybridization conditions.

High Stringency condition (detects sequences that share at least 90% sequence identity): Hybridization in 5×SSC buffer at 65° C. for 16 hours; wash twice in 2×SSC buffer at room temperature for 15 minutes each; and wash twice in 0.5×SSC buffer at 65° C. for 20 minutes each.

Moderate Stringency condition (detects sequences that share at least 80% sequence identity): Hybridization in 5×-6×SSC buffer at 65-70° C. for 16-20 hours; wash twice in 2×SSC buffer at room temperature for 5-20 minutes each; and wash twice in 1×SSC buffer at 55-70° C. for 30 minutes each.

Non-stringent control condition (sequences that share at least 50% sequence identity will hybridize): Hybridization in 6×SSC buffer at room temperature to 55° C. for 16-20 hours; wash at least twice in 2×-3×SSC buffer at room temperature to 55° C. for 20-30 minutes each.

As used herein, the term “substantially homologous” or “substantial homology,” with regard to a polynucleotide, refers to polynucleotides that hybridize under stringent conditions to the reference nucleic acid sequence. For example, polynucleotides that are substantially homologous to a reference DNA coding sequence are those polynucleotides that hybridize under stringent conditions (e.g., the Moderate Stringency conditions set forth, supra) to the reference DNA coding sequence. Substantially homologous sequences may have at least 80% sequence identity. For example, substantially homologous sequences may have from about 80% to 100% sequence identity, such as about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; and about 100%. The property of substantial homology is closely related to specific hybridization. For example, a nucleic acid molecule is specifically hybridizable when there is a sufficient degree of complementarity to avoid non-specific binding of the nucleic acid to non-target sequences under conditions where specific binding is desired, for example, under stringent hybridization conditions.

As used herein, the term “ortholog” refers to a gene in two or more species that has evolved from a common ancestral nucleotide sequence, and may retain the same function in the two or more species.

As used herein, two nucleic acid sequence molecules are said to exhibit “complete complementarity” when every nucleotide of a sequence read in the 5′ to 3′ direction is complementary to every nucleotide of the other sequence when read in the 3′ to 5′ direction. A nucleotide sequence that is complementary to a reference nucleotide sequence will exhibit a sequence identical to the reverse complement sequence of the reference nucleotide sequence. These terms and descriptions are well defined in the art and are easily understood by those of ordinary skill in the art.

As used herein, the term “substantially identical” may refer to nucleotide sequences that are more than 85% identical. For example, a substantially identical nucleotide sequence may be at least 85.5%; at least 86%; at least 87%; at least 88%; at least 89%; at least 90%; at least 91%; at least 92%; at least 93%; at least 94%; at least 95%; at least 96%; at least 97%; at least 98%; at least 99%; or at least 99.5% identical to the reference sequence.

Expression: As used herein, “expression” of a coding sequence (for example, a gene or a transgene) refers to the process by which the coded information of a nucleic acid transcriptional unit (including, e.g., genomic DNA or cDNA) is converted into an operational, non-operational, or structural part of a cell (e.g., a protein). Gene expression can be influenced by external signals; for example, exposure of a cell, tissue, or organism to an agent that increases or decreases expression of a gene comprised therein. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, and/or through activation, inactivation, compartmentalization, or degradation of specific protein molecules after they have been made, or by combinations of any of the foregoing. Gene expression can be measured at the RNA level or the protein level by methods known in the art, including, without limitation, Northern blot, RT-PCR, Western blot, and in vitro, in situ, or in vivo protein activity assay(s).

Increase expression: As used herein, the term “increase expression” refers to initiation of expression, as well as to a quantitative increase in the amount of an expression product produced from a template construct. In some embodiments, at least one heterologous gene may be provided to a cell or organism that otherwise comprises an endogenous copy of the same gene, so as to increase the expression of the polypeptide encoded by the gene. In such embodiments, the increase in expression may be determined by comparison of the amount of the polypeptide produced in the cell comprising the heterologous and endogenous genes, with the amount produced in the cell comprising only the endogenous gene. In some embodiments, a first polypeptide that affects transcription (e.g., NSP1-like and/or NSP2-like) may be provided to a cell or organism, so as to increase the expression of a second polypeptide encoded by a gene under the control of the first polypeptide. In such embodiments, the increase in expression may be determined by comparison of the amount of the polypeptide produced from the gene in the presence of the first polypeptide, with the amount produced from the gene in the absence of the first polypeptide. In some embodiments, a regulatory sequence may be operably linked to a gene, so as to increase the expression of the gene. In such embodiments, the increase in expression may be determined by comparison of the amount of the polypeptide produced from the gene after operable linkage of the regulatory sequence thereto, with the amount produced from the gene before operable linkage or introduction of the regulatory sequence.

Operably linked: A first nucleotide sequence is operably linked with a second nucleic acid sequence when the first nucleic acid sequence is in a functional relationship with the second nucleic acid sequence. When recombinantly produced, operably linked nucleic acid sequences are generally contiguous, and, where necessary to join two protein-coding regions, in the same reading frame (e.g., in a polycistronic ORF). However, nucleic acids need not be contiguous to be operably linked.

The term, “operably linked,” when used in reference to a regulatory sequence and a coding sequence, means that the regulatory sequence affects the expression of the linked coding sequence. “Regulatory sequences,” or “control elements,” refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

Promoter: As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell.

Some embodiments herein include a “plant promoter.” A plant promoter is a promoter that is capable of initiating transcription in a plant cell.

Some embodiments herein include a “tissue-preferred promoter.” A tissue-preferred promoter is a promoter that is capable of initiating transcription under developmental control, and include, for example and without limitation: promoters that preferentially initiate transcription in leaves, pollen, tassels, roots, seeds, fibers, xylem vessels, tracheids, and sclerenchyma. Promoters that initiate transcription essentially only in certain tissues are referred to as “tissue-specific.” A “cell type-specific” promoter primarily drives expression in certain cell types in one or more organs, for example, vascular cells in roots or leaves. An “inducible” promoter may be a promoter which may be under environmental control. Examples of environmental conditions that may initiate transcription by inducible promoters include anaerobic conditions and the presence of light. Tissue-specific, tissue-preferred, cell type specific, and inducible promoters constitute the class of “non-constitutive” promoters.

Any inducible promoter may be used in some embodiments herein. See Ward et al. (1993) Plant Mol. Biol. 22:361-366. With an inducible promoter, the rate of transcription increases in response to an inducing agent. Exemplary inducible promoters include, but are not limited to: Promoters from the ACEI system that responds to copper; In2 gene from maize that responds to benzenesulfonamide herbicide safeners; Tet repressor from Tn10; and the inducible promoter from a steroid hormone gene, the transcriptional activity of which may be induced by a glucocorticosteroid hormone (Schena et al. (1991) Proc. Natl. Acad. Sci. USA 88:10421-5).

In contrast to non-constitutive promoters, a “constitutive” promoter is a promoter that is active under most environmental conditions. Exemplary constitutive promoters include, but are not limited to: promoters from plant viruses, such as the 35S promoter from CaMV; promoters from rice actin genes; ubiquitin promoters; pEMU; MAS; maize H3 histone promoter; and the ALS promoter, Xba1/NcoI fragment 5′ to the Brassica napus ALS3 structural gene (or a nucleotide sequence similarity to said Xba1/NcoI fragment) (PCT International Patent Publication No. WO 96/30530).

Additionally, any tissue-specific or tissue-preferred promoter may be utilized in some embodiments of the invention. Plants transformed with a nucleic acid molecule comprising a coding sequence operably linked to a tissue-specific promoter may produce the product of the coding sequence exclusively, or preferentially, in a specific tissue. Exemplary tissue-specific or tissue-preferred promoters include, but are not limited to: a root-preferred promoter, such as that from the phaseolin gene; a leaf-specific and light-induced promoter such as that from cab or rubisco; an anther-specific promoter such as that from LAT52; a pollen-specific promoter such as that from Zm13; and a microspore-preferred promoter such as that from apg.

Conservative substitution: As used herein, the term “conservative substitution” refers to a substitution where an amino acid residue is substituted for another amino acid in the same class. A non-conservative amino acid substitution is one where the residues do not fall into the same class, for example, substitution of a basic amino acid for a neutral or non-polar amino acid. Classes of amino acids that may be defined for the purpose of performing a conservative substitution are known in the art.

In some embodiments, a conservative substitution includes the substitution of a first aliphatic amino acid for a second, different aliphatic amino acid. For example, if a first amino acid is one of Gly; Ala; Pro; Ile; Leu; Val; and Met, the first amino acid may be replaced by a second, different amino acid selected from Gly; Ala; Pro; Ile; Leu; Val; and Met. In particular examples, if a first amino acid is one of Gly; Ala; Pro; Ile; Leu; and Val, the first amino acid may be replaced by a second, different amino acid selected from Gly; Ala; Pro; Ile; Leu; and Val. In particular examples involving the substitution of hydrophobic aliphatic amino acids, if a first amino acid is one of Ala; Pro; Ile; Leu; and Val, the first amino acid may be replaced by a second, different amino acid selected from Ala; Pro; Ile; Leu; and Val.

In other embodiments, a conservative substitution includes the substitution of a first aromatic amino acid for a second, different aromatic amino acid. For example, if a first amino acid is one of His; Phe; Trp; and Tyr, the first amino acid may be replaced by a second, different amino acid selected from His; Phe; Trp; and Tyr. In particular examples involving the substitution of uncharged aromatic amino acids, if a first amino acid is one of Phe; Trp; and Tyr, the first amino acid may be replaced by a second, different amino acid selected from Phe; Trp; and Tyr.

In alternative embodiments, a conservative substitution includes the substitution of a first hydrophobic amino acid for a second, different hydrophobic amino acid. For example, if a first amino acid is one of Ala; Val; Ile; Leu; Met; Phe; Tyr; and Trp, the first amino acid may be replaced by a second, different amino acid selected from Ala; Val; Ile; Leu; Met; Phe; Tyr; and Trp. In particular examples involving the substitution of non-aromatic, hydrophobic amino acids, if a first amino acid is one of Ala; Val; Ile; Leu; and Met, the first amino acid may be replaced by a second, different amino acid selected from Ala; Val; Ile; Leu; and Met.

In some embodiments, a conservative substitution includes the substitution of a first polar amino acid for a second, different polar amino acid. For example, if a first amino acid is one of Ser; Thr; Asn; Gln; Cys; Gly; Pro; Arg; His; Lys; Asp; and Glu, the first amino acid may be replaced by a second, different amino acid selected from Ser; Thr; Asn; Gln; Cys; Gly; Pro; Arg; His; Lys; Asp; and Glu. In particular examples involving the substitution of uncharged, polar amino acids, if a first amino acid is one of Ser; Thr; Asn; Gln; Cys; Gly; and Pro, the first amino acid may be replaced by a second, different amino acid selected from Ser; Thr; Asn; Gln; Cys; Gly; and Pro. In particular examples involving the substitution of charged, polar amino acids, if a first amino acid is one of His; Arg; Lys; Asp; and Glu, the first amino acid may be replaced by a second, different amino acid selected from His; Arg; Lys; Asp; and Glu. In further examples involving the substitution of charged, polar amino acids, if a first amino acid is one of Arg; Lys; Asp; and Glu, the first amino acid may be replaced by a second, different amino acid selected from Arg; Lys; Asp; and Glu. In particular examples involving the substitution of positively charged (basic), polar amino acids, if a first amino acid is one of His; Arg; and Lys, the first amino acid may be replaced by a second, different amino acid selected from His; Arg; and Lys. In further examples involving the substitution of positively charged, polar amino acids, if a first amino acid is Arg or Lys, the first amino acid may be replaced by the other amino acid of Arg and Lys. In particular examples involving the substitution of negatively charged (acidic), polar amino acids, if a first amino acid is Asp or Glu, the first amino acid may be replaced by the other amino acid of Asp and Glu.

In additional embodiments, a conservative substitution includes the substitution of a first electrically neutral amino acid for a second, different electrically neutral amino acid. For example, if a first amino acid is one of Gly; Ser; Thr; Cys; Asn; Gln; and Tyr, the first amino acid may be replaced by a second, different amino acid selected from Gly; Ser; Thr; Cys; Asn; Gln; and Tyr.

In some embodiments, a conservative substitution includes the substitution of a first non-polar amino acid for a second, different non-polar amino acid. For example, if a first amino acid is one of Ala; Val; Leu; Ile; Phe; Trp; Pro; and Met, the first amino acid may be replaced by a second, different amino acid selected from Ala; Val; Leu; Ile; Phe; Trp; Pro; and Met.

In many examples, the selection of a particular second amino acid to be used in a conservative substitution to replace a first amino acid may be made in order to maximize the number of the foregoing classes to which the first and second amino acids both belong. Thus, if the first amino acid is Ser (a polar, non-aromatic, and electrically neutral amino acid), the second amino acid may be another polar amino acid (i.e., Thr; Asn; Gln; Cys; Gly; Pro; Arg; His; Lys; Asp; or Glu); another non-aromatic amino acid (i.e., Thr; Asn; Gln; Cys; Gly; Pro; Arg; His; Lys; Asp; Glu; Ala; Ile; Leu; Val; or Met); or another electrically-neutral amino acid (i.e., Gly; Thr; Cys; Asn; Gln; or Tyr). However, it may be preferred that the second amino acid in this case be one of Thr; Asn; Gln; Cys; and Gly, because these amino acids share all the classifications according to polarity, non-aromaticity, and electrical neutrality. Additional criteria that may optionally be used to select a particular second amino acid to be used in a conservative substitution are known in the art. For example, when Thr; Asn; Gln; Cys; and Gly are available to be used in a conservative substitution for Ser, Cys may be eliminated from selection in order to avoid the formation of undesirable cross-linkages and/or disulfide bonds. Likewise, Gly may be eliminate from selection, because it lacks an alkyl side chain. In this case, Thr may be selected, e.g., in order to retain the functionality of a side chain hydroxyl group. The selection of the particular second amino acid to be used in a conservative substitution is ultimately, however, within the discretion of the skilled practitioner.

Nitrogen-limiting conditions: As used herein, the term “nitrogen-limiting conditions” refers to conditions wherein there is a limited amount of nitrogen sources (e.g., nitrate and ammonium) in the soil or culture medium. The amount that is “limiting” is in some examples a range of nitrogen concentration from 0.0 to 0.2 mM; e.g., from 0 to 0.1 mM, from 0 to 0.03 mM, and from 0 to 0.05 mM.

Trait or phenotype: The terms “trait” and “phenotype” are used interchangeably herein. For the purposes of the present disclosure, traits of particular interest include agronomically important traits, as may be expressed, for example, in a crop plant.

Transformation: As used herein, the term “transformation” refers to the transfer of one or more nucleic acid molecule(s) into a cell. A cell is “transformed” by a nucleic acid molecule introduced into the cell when the nucleic acid molecule becomes stably replicated by the cell, either by incorporation of the nucleic acid molecule into the cellular genome, or by episomal replication. As used herein, the term “transformation” encompasses all techniques by which a nucleic acid molecule can be introduced into such a cell. Examples include, but are not limited to: transfection with viral vectors; transformation with plasmid vectors; electroporation (Fromm et al. (1986) Nature 319:791-3); lipofection (Felgner et al. (1987) Proc. Natl. Acad. Sci. USA 84:7413-7); microinjection (Mueller et al. (1978) Cell 15:579-85); Agrobacterium-mediated transfer (Fraley et al. (1983) Proc. Natl. Acad. Sci. USA 80:4803-7); direct DNA uptake; and microprojectile bombardment (Klein et al. (1987) Nature 327:70).

Transgene: A transgene is an exogenous nucleic acid sequence. In some examples, a transgene may be a sequence that encodes one or both strand(s) of a dsRNA molecule that comprises a nucleotide sequence that is complementary to a target nucleic acid. In some examples, a transgene may be an antisense nucleic acid sequence, the expression of which inhibits expression of a target nucleic acid. In still other examples, a transgene may be a gene sequence (e.g., a herbicide-resistance gene), a gene encoding an industrially or pharmaceutically useful compound, or a gene encoding a desirable agricultural trait. In these and other examples, a transgene may contain regulatory sequences operably linked to the coding sequence of the transgene (e.g., a promoter).

Vector: A vector refers to a nucleic acid molecule as introduced into a cell, for example, to produce a transformed cell. A vector may include nucleic acid sequences that permit it to replicate in the host cell, such as an origin of replication. Examples of vectors include, but are not limited to: a plasmid; cosmid; bacteriophage; and a virus that carries exogenous DNA into a cell. A vector may also include one or more genes, antisense molecules, and/or selectable marker genes and other genetic elements known in the art. A vector may transduce, transform, or infect a cell, thereby causing the cell to express the nucleic acid molecules and/or proteins encoded by the vector. A vector optionally includes materials to aid in achieving entry of the nucleic acid molecule into the cell (e.g., a liposome, protein coating, etc.).

Unless specifically indicated or implied, the terms “a”, “an”, and “the” signify “at least one,” as used herein.

Unless otherwise specifically explained, all technical and scientific terms used herein have the same meaning as commonly understood by those of ordinary skill in the art to which this disclosure belongs. Definitions of common terms in molecular biology can be found in, for example, Lewin B., Genes V, Oxford University Press, 1994 (ISBN 0-19-854287-9); Kendrew et al. (eds.), The Encyclopedia of Molecular Biology, Blackwell Science Ltd., 1994 (ISBN 0-632-02182-9); and Meyers R. A. (ed.), Molecular Biology and Biotechnology: A Comprehensive Desk Reference, VCH Publishers, Inc., 1995 (ISBN 1-56081-569-8). All percentages are by weight and all solvent mixture proportions are by volume unless otherwise noted. All temperatures are in degrees Celsius.

### IV. BNF Genes in Non-Nodulating Plants

This disclosure provides compositions and methods that exploit a new and unexpected use for the transcription factors, NSP1-like and NSP2-like, and NIN-like genes (e.g., NLPs1-9). As disclosed herein, NSP1-like, NLP4, and NLP9 influence the functional interaction between plants (e.g., non-nodulating plants, such as Arabidopsis) and NFB (e.g., S. meliloti, such as RMP110). Thus, for example, NSP1-like, NLP4, and/or NLP9 may be used to regulate the association of a NFB with a crop plant. The properties of NSP1-like, NLP4, and NLP9 described herein may be used, for example, to provide transgenic plants with an altered BNF phenotype. For example, NSP1-like (SCL29), NLP4, and/or NLP9 may be expressed or over-expressed in a plant to initiate and/or increase the ability of the plant to associate with NFB under N-limiting conditions, and/or to increase the efficiency of the plant's utilization of environmental nitrogen. In certain examples, NSP1-like (SCL29), NLP4, and/or NLP9 may be introduced into a plant or over-expressed in a plant that normally comprises the polypeptide (e.g., by introducing additional copies of a gene encoding the polypeptide, and/or changing the regulatory control of the normally present gene).

Some embodiments include a NSP1-like transcription factor polypeptide. NSP1-like polypeptides according to particular embodiments comprise an amino acid sequence showing increasing percentage identities when aligned with SEQ ID NO:1 (Arabidopsis thaliana NSP1-like). Specific amino acid sequences within these and other embodiments may comprise sequences having, for example, at least about 50%, about 55%, about 60%, about 65%, about 70%, about 75%, about 80%, 81%, 82%, 83%, 84%, 85%, 86%, 87%, 88%, 89%, 90%, 91%, 92%, 93%, 94%, 95% 96%, 97%, 98%, 99%, or 100% identity with SEQ ID NO:1.

Other embodiments include an NIN-like protein polypeptide. Particular embodiments include an NLP4 polypeptide, and/or an NLP9 polypeptide.

NLP4 polypeptides according to some examples comprise an amino acid sequence showing increasing percentage identities when aligned with SEQ ID NO:2 (A. thaliana NLP4). Specific amino acid sequences within these and other embodiments may comprise sequences having, for example, at least about 50%, about 55%, about 60%, about 65%, about 70%, about 75%, about 80%, 81%, 82%, 83%, 84%, 85%, 86%, 87%, 88%, 89%, 90%, 91%, 92%, 93%, 94%, 95% 96%, 97%, 98%, 99%, or 100% identity with SEQ ID NO:2. For example, some embodiments include an AtNLP4 ortholog.

NLP9 polypeptides according to some examples comprise an amino acid sequence showing increasing percentage identities when aligned with SEQ ID NO:3 (A. thaliana NLP9). Specific amino acid sequences within these and other embodiments may comprise sequences having, for example, at least about 50%, about 55%, about 60%, about 65%, about 70%, about 75%, about 80%, 81%, 82%, 83%, 84%, 85%, 86%, 87%, 88%, 89%, 90%, 91%, 92%, 93%, 94%, 95% 96%, 97%, 98%, 99%, or 100% identity with SEQ ID NO:9. For example, some embodiments include an AtNLP9 ortholog.

In some embodiments, a polypeptide comprising an amino acid sequence having the aforementioned sequence identity when aligned with SEQ ID NO:1 (NSP1-like polypeptides) or SEQ ID NO:2 (NLP4 polypeptides) and/or SEQ ID NO:3 (NLP9 polypeptides) is comprised within a protein that is required for the association of a plant comprising the protein and NFB. NSP1-like polypeptides may be identified, for example, by searching a sequence database for polypeptide sequences having a threshold sequence identity with SEQ ID NO:1. NLP4 polypeptides may be identified, for example, by searching a sequence database for polypeptide sequences having a certain sequence identity with SEQ ID NO:2. NLP9 polypeptides may be identified, for example, by searching a sequence database for polypeptide sequences having a certain sequence identity with SEQ ID NO:3. Useful sequence databases may be searched by any of many methods known to those of skill in the art (e.g., utilizing NCBI's BLAST® tool). Other databases are available for many plants and other organisms through a variety of public and private commercial sources. As will be appreciated by those of skill in the art, NLP4 and NLP9 are homologous proteins, and thus, a particular polypeptide identified as comprising an amino acid sequence sharing sequence identity with SEQ ID NO:2 or SEQ ID NO:3 may also share sequence identity with the other of SEQ ID NOs:2 and 4.

Other embodiments include a structural equivalent of an NSP1-like polypeptide and/or a structural equivalent of an NLP4 polypeptide and/or an NLP9 polypeptide. Structural equivalents include, but are not limited to, conservative substitutions of amino acid residues within the amino acid sequence of the NSP1-like, NLP4, and NLP9 polypeptides herein. As used herein, a “conservative substitution” is a substitution where an amino acid residue is substituted for another amino acid in the same class. A non-conservative amino acid substitution is one where the residues do not fall into the same class, for example, substitution of a basic amino acid for a neutral or non-polar amino acid. Classes of amino acids that may be defined for the purpose of performing a conservative substitution are known in the art.

In alternative embodiments, a conservative substitution includes the substitution of a first aliphatic amino acid for a second, different aliphatic amino acid. For example, if a first amino acid is one of Gly; Ala; Pro; Ile; Leu; Val; and Met, the first amino acid may be replaced by a second, different amino acid selected from Gly; Ala; Pro; Ile; Leu; Val; and Met. In particular examples, if a first amino acid is one of Gly; Ala; Pro; Ile; Leu; and Val, the first amino acid may be replaced by a second, different amino acid selected from Gly; Ala; Pro; Ile; Leu; and Val. In particular examples involving the substitution of hydrophobic aliphatic amino acids, if a first amino acid is one of Ala; Pro; Ile; Leu; and Val, the first amino acid may be replaced by a second, different amino acid selected from Ala; Pro; Ile; Leu; and Val.

In other embodiments, a conservative substitution includes the substitution of a first aromatic amino acid for a second, different aromatic amino acid. For example, if a first amino acid is one of His; Phe; Trp; and Tyr, the first amino acid may be replaced by a second, different amino acid selected from His; Phe; Trp; and Tyr. In particular examples involving the substitution of uncharged aromatic amino acids, if a first amino acid is one of Phe; Trp; and Tyr, the first amino acid may be replaced by a second, different amino acid selected from Phe; Trp; and Tyr.

In some embodiments, a conservative substitution includes the substitution of a first hydrophobic amino acid for a second, different hydrophobic amino acid. For example, if a first amino acid is one of Ala; Val; Ile; Leu; Met; Phe; Tyr; and Trp, the first amino acid may be replaced by a second, different amino acid selected from Ala; Val; Ile; Leu; Met; Phe; Tyr; and Trp. In particular examples involving the substitution of non-aromatic, hydrophobic amino acids, if a first amino acid is one of Ala; Val; Ile; Leu; and Met, the first amino acid may be replaced by a second, different amino acid selected from Ala; Val; Ile; Leu; and Met.

In additional embodiments, a conservative substitution includes the substitution of a first polar amino acid for a second, different polar amino acid. For example, if a first amino acid is one of Ser; Thr; Asn; Gln; Cys; Gly; Pro; Arg; His; Lys; Asp; and Glu, the first amino acid may be replaced by a second, different amino acid selected from Ser; Thr; Asn; Gln; Cys; Gly; Pro; Arg; His; Lys; Asp; and Glu. In particular examples involving the substitution of uncharged, polar amino acids, if a first amino acid is one of Ser; Thr; Asn; Gln; Cys; Gly; and Pro, the first amino acid may be replaced by a second, different amino acid selected from Ser; Thr; Asn; Gln; Cys; Gly; and Pro. In particular examples involving the substitution of charged, polar amino acids, if a first amino acid is one of His; Arg; Lys; Asp; and Glu, the first amino acid may be replaced by a second, different amino acid selected from His; Arg; Lys; Asp; and Glu. In further examples involving the substitution of charged, polar amino acids, if a first amino acid is one of Arg; Lys; Asp; and Glu, the first amino acid may be replaced by a second, different amino acid selected from Arg; Lys; Asp; and Glu. In particular examples involving the substitution of positively charged (basic), polar amino acids, if a first amino acid is one of His; Arg; and Lys, the first amino acid may be replaced by a second, different amino acid selected from His; Arg; and Lys. In further examples involving the substitution of positively charged, polar amino acids, if a first amino acid is Arg or Lys, the first amino acid may be replaced by the other amino acid of Arg and Lys. In particular examples involving the substitution of negatively charged (acidic), polar amino acids, if a first amino acid is Asp or Glu, the first amino acid may be replaced by the other amino acid of Asp and Glu.

In some embodiments, a conservative substitution includes the substitution of a first electrically neutral amino acid for a second, different electrically neutral amino acid. For example, if a first amino acid is one of Gly; Ser; Thr; Cys; Asn; Gln; and Tyr, the first amino acid may be replaced by a second, different amino acid selected from Gly; Ser; Thr; Cys; Asn; Gln; and Tyr.

In further embodiments, a conservative substitution includes the substitution of a first non-polar amino acid for a second, different non-polar amino acid. For example, if a first amino acid is one of Ala; Val; Leu; Ile; Phe; Trp; Pro; and Met, the first amino acid may be replaced by a second, different amino acid selected from Ala; Val; Leu; Ile; Phe; Trp; Pro; and Met.

In many examples, the selection of a particular second amino acid to be used in a conservative substitution to replace a first amino acid may be made in order to maximize the number of the foregoing classes to which the first and second amino acids both belong. Thus, if the first amino acid is Ser (a polar, non-aromatic, and electrically neutral amino acid), the second amino acid may be another polar amino acid (i.e., Thr; Asn; Gln; Cys; Gly; Pro; Arg; His; Lys; Asp; or Glu); another non-aromatic amino acid (i.e., Thr; Asn; Gln; Cys; Gly; Pro; Arg; His; Lys; Asp; Glu; Ala; Ile; Leu; Val; or Met); or another electrically-neutral amino acid (i.e., Gly; Thr; Cys; Asn; Gln; or Tyr). However, it may be preferred that the second amino acid in this case be one of Thr; Asn; Gln; Cys; and Gly, because these amino acids share all the classifications according to polarity, non-aromaticity, and electrical neutrality. Additional criteria that may optionally be used to select a particular second amino acid to be used in a conservative substitution are known in the art. For example, when Thr; Asn; Gln; Cys; and Gly are available to be used in a conservative substitution for Ser, Cys may be eliminated from selection in order to avoid the formation of undesirable cross-linkages and/or disulfide bonds. Likewise, Gly may be eliminate from selection, because it lacks an alkyl side chain. In this case, Thr may be selected, e.g., in order to retain the functionality of a side chain hydroxyl group. The selection of the particular second amino acid to be used in a conservative substitution is ultimately, however, within the discretion of the skilled practitioner.

Other mutations to the plant NSP1-like, NLP4, and NLP9 coding polynucleotides can be made to generate expression products that are better suited for, for example, expression and scale up, in a host cell comprising the polynucleotide(s). For example, cysteine residues can be deleted or substituted with another amino acid in order to eliminate disulfide bridges; N-linked glycosylation sites can be altered or eliminated to achieve, for example, expression of a homogeneous product that is more easily recovered and purified from yeast hosts which are known to hyperglycosylate N-linked sites. To this end, a variety of amino acid substitutions at one or both of the first or third amino acid positions of any one or more of the glycosylation recognition sequences which occur in the extracellular domain (ECD) (N-X-S or N-X-T), and/or an amino acid deletion at the second position of any one or more such recognition sequences in the ECD will prevent glycosylation of the mutant polypeptide at the modified tripeptide sequence.

Hydrophilic amino acids generally include and generally have the respective relative degree of hydrophobicity (at pH 7.0; kcal/mol) as follows: aspartic acid (D), −7.4; glutamic acid (E) −9.9; asparagine (N), −0.2; glutamine (Q), −0.3; lysine (K), −4.2; arginine (R), −11.2; serine (S), −0.3; and cysteine (C), −2.8. Hydrophobic amino acids generally include and generally have the respective relative degree of hydrophobicity as follows: histidine (H), 0.5; threonine (T), 0.4; tyrosine (Y), 2.3; tryptophan (W), 3.4; phenylalanine (F), 2.5; leucine (L), 1.8; isoleucine (I), 2.5; methionine (M), 1.3; valine (V), 1.5; and alanine (A), 0.5. Glycine has a relative degree of hydrophobicity of 0 and may be considered to be hydrophilic or hydrophobic.

The amino acid homology of peptides can be readily determined by contrasting the amino acid sequences thereof as is known in the art. Similarly, the amphiphilic homology of peptides can be determined by contrasting the hydrophilicity and hydrophobicity of the amino acid sequences.

Some embodiments include a nucleic acid comprising a nucleotide sequence encoding a NSP1-like (a “NSP1-like polynucleotide”), NLP4 (a “NLP4 polynucleotide”), and/or NLP9 polypeptide (a “NLP9 polynucleotide”), such as are described above. For example, nucleic acid sequences in some embodiments show increasing percentage identities when aligned with SEQ ID NO:4 (A. thaliana NSP1-like) or SEQ ID NO:5 (A. thaliana NLP4) and/or SEQ ID NO:6 (A. thaliana NLP9). Specific nucleic acid sequences within these and other embodiments may comprise sequences having, for example and without limitation, at least about 50%, about 55%, about 60%, about 65%, about 70%, about 75%, about 80%, 81%, 82%, 83%, 84%, 85%, 86%, 87%, 88%, 89%, 90%, 91%, 92%, 93%, 94%, 95% 96%, 97%, 98%, 99%, or 100% identity SEQ ID NO:4 or SEQ ID NO:5 and/or SEQ ID NO:6.

A large number of nucleic acids comprising a nucleotide sequence encoding a NSP1-like, NLP4, and/or NLP9 polypeptide can be readily identified by those of skill in the art. For example, nucleic acid molecules may be modified without substantially changing the amino acid sequence of the encoded polypeptide, for example, by introducing permissible nucleotide substitutions according to codon degeneracy. Thus, it will be understood that any NSP1-like, NLP4, and/or NLP9 polypeptide with a given amino acid sequence may be immediately reverse-engineered to any of many redundant nucleotide sequences. By way of further example, genes encoding a NSP1-like, NLP4, and/or NLP9 polypeptide may be selected from any of the many available plant genomic libraries, cDNA libraries, EST libraries, and the like (e.g., by homology to one of SEQ ID NOs:4-6), or by sequence similarity of an encoded polypeptide with SEQ ID NO:1 or SEQ ID NOs:2 and/or 3, or such genes may be cloned from an organism according to reliable and well-known techniques in molecular biology.

Any and all NSP1-like, NLP4, and NLP9 polypeptides, and nucleic acid molecules encoding either of the same, may be utilized in certain embodiments of the invention.

In some embodiments herein, a nucleic acid comprising a nucleotide sequence encoding a NSP1-like, NLP4, and/or NLP9 polypeptide comprises a gene regulatory element (e.g., a promoter). Promoters may be selected on the basis of the cell type into which the vector construct will be inserted. Promoters which function in bacteria, yeast, and plants are well-known in the art. The promoters may also be selected on the basis of their regulatory features. Examples of such features include enhancement of transcriptional activity, inducibility, tissue-specificity, and developmental stage-specificity. In plants, promoters that are inducible, of viral or synthetic origin, constitutively active, temporally regulated, and spatially regulated have been described. See, e.g., Poszkowski et al. (1989) EMBO J. 3:2719; Odell et al. (1985) Nature 313:810; and Chau et al. (1989) Science 244:174-81).

To obtain higher expression of a heterologous gene(s), it may be preferred to reengineer the gene(s) so that it is more efficiently expressed in the expression host cell (e.g., a plant cell, for example, canola, rice, tobacco, maize, cotton, and soybean). Therefore, an optional additional step in the design of a gene encoding a NSP1-like or NLP4 and/or NLP9 polypeptide for plant expression (i.e., in addition to the provision of one or more gene regulatory elements) is reengineering of a heterologous gene protein coding region for optimal expression. Particular examples include a redesigned Arabidopsis gene that has been optimized to increase the expression level (i.e. produce more protein) in a transgenic plant cell from a second plant species than in a plant cell from the second plant species transformed with the original (i.e., unmodified) Arabidopsis gene sequence.

Due to the plasticity afforded by the redundancy/degeneracy of the genetic code (i.e., some amino acids are specified by more than one codon), evolution of the genomes in different organisms or classes of organisms has resulted in differential usage of synonymous codons. This “codon bias” is reflected in the mean base composition of protein coding regions. For example, organisms having genomes with relatively low G+C contents utilize more codons having A or T in the third position of synonymous codons, whereas those having higher G+C contents utilize more codons having G or C in the third position. Further, it is thought that the presence of “minor” codons within an mRNA may reduce the absolute translation rate of that mRNA, especially when the relative abundance of the charged tRNA corresponding to the minor codon is low. An extension of this reasoning is that the diminution of translation rate by individual minor codons would be at least additive for multiple minor codons. Therefore, mRNAs having high relative contents of minor codons in a particular expression host would have correspondingly low translation rates. This rate may be reflected by correspondingly low levels of the encoded protein.

In engineering optimized genes encoding a NSP1-like or NLP4 and/or NLP9 polypeptide for expression in a plant cell (e.g., rice, tobacco, maize, cotton, and soybean), it is helpful if the codon bias of the prospective host plant(s) has been determined. Multiple publicly-available DNA sequence databases exist wherein one may find information about the codon distribution of plant genomes or the protein coding regions of various plant genes.

The codon bias is the statistical distribution of codons that the expression host uses for coding the amino acids of its proteins. The codon bias can be calculated as the frequency at which a single codon is used relative to the codons for all amino acids. Alternatively, the codon bias may be calculated as the frequency at which a single codon is used to encode a particular amino acid, relative to all the other codons for that amino acid (synonymous codons).

In designing optimized coding regions for plant expression of NSP1-like or NLP4 and/or NLP9 polypeptides, the primary (“first choice”) codons preferred by the plant should be determined, as well as the second, third, fourth etc. choices of preferred codons when multiple choices exist. A new DNA sequence can then be designed which encodes the amino sequence of the NSP1-like or NLP4 and/or NLP9 polypeptide, wherein the new DNA sequence differs from the native DNA sequence (encoding the polypeptide) by the substitution of expression host-preferred (first preferred, second preferred, third preferred, or fourth preferred, etc.) codons to specify the amino acid at each position within the amino acid sequence. The new sequence is then analyzed for restriction enzyme sites that might have been created by the modifications. The identified putative restriction sites are further modified by replacing these codons with a next-preferred codon to remove the restriction site. Other sites in the sequence which may affect transcription or translation of heterologous sequence are exon:intron junctions (5′ or 3′), poly-A addition signals, and/or RNA polymerase termination signals. The sequence may be further analyzed and modified to reduce the frequency of TA or CG doublets. In addition to these doublets, sequence blocks that have more than about six G or C nucleotides that are the same may also adversely affect transcription or translation of the sequence. Therefore, these blocks are advantageously modified by replacing the codons of first or second choice, etc. with the next-preferred codon of choice.

A method such as that described above enables one skilled in the art to modify gene(s) that are foreign to a particular plant so that the genes are optimally expressed in plants. The method is further illustrated in PCT International Patent Publication No. WO 97/13402 A1. Thus, optimized synthetic genes that are functionally equivalent to NSP1-like, NLP4, and/or NLP9 polynucleotides of some embodiments may be used to transform hosts, including plants and plant cells. Furthermore, NSP1-like, NLP4, and NLP9 polynucleotides may also be generated, in silico, from an initial amino acid sequence. Additional guidance regarding the production of synthetic genes can be found in, for example, U.S. Pat. No. 5,380,831.

Once a NSP1-like, NLP4, or NLP9 polynucleotide sequence has been designed on paper or in silico, actual nucleic acid molecules comprising the polynucleotide sequence can be synthesized in the laboratory to correspond in sequence precisely to the designed sequence. Such synthetic DNA molecules may be cloned and otherwise manipulated exactly as if they were derived from natural or native sources.

### V. Alteration of Plant Growth in N-Limiting Conditions by NSP1-Like, NLP4, and/or NLP9

Some embodiments exploit the discovery that NSP1-like, NLP4, and NLP9 are necessary for plants to maintain and/or increase growth through an association with NFB in N-limiting growth conditions. For example, NSP1-like, NLP4, and/or NLP9 polypeptides may be introduced or heterologously expressed in a plant cell, for example and without limitation, to increase the association of a plant comprising the plant cell with NFB; to increase the growth of the plant under N-limited conditions; and to increase the efficiency with which the plant utilizes environmental nitrogen.

In particular embodiments, a NSP1-like, NLP4, and/or NLP9 polypeptide may be expressed or over-expressed in a cell or organism, for example and without limitation, by introducing a NSP1-like, NLP4, and/or NLP9 polynucleotide into the cell or organism; by introducing the NSP1-like, NLP4, and/or NLP9 polypeptide into the cell or organism; and/or by providing positive or negative signals sufficient to promote expression of the NSP1-like, NLP4, and/or NLP9 polypeptide through an interaction of the signal(s) with regulatory elements operably linked to a NSP1-like, NLP4, and/or NLP9 polynucleotide in the cell or organism. In further embodiments, a NSP1-like, NLP4, and/or NLP9 polypeptide may be knocked-out or under-expressed in a cell or organism, for example and without limitation, by disrupting, mutating, or inactivating a NSP1-like, NLP4, and/or NLP9 polynucleotide; introducing an antisense nucleic acid into the cell or organism that targets a NSP1-like, NLP4, and/or NLP9 polynucleotide; by physically removing the NSP1-like, NLP4, and/or NLP9 polypeptide from the cellular machinery of the cell or organism by binding the NSP1-like, NLP4, and/or NLP9 polypeptide with antibodies or other specific binding proteins; and/or by providing positive or negative signals sufficient to reduce or eliminate expression of the NSP1-like, NLP4, and/or NLP9 polypeptide through an interaction of the signal(s) with regulatory elements operably linked to a NSP1-like, NLP4, and/or NLP9 polynucleotide in the cell or organism.

In some embodiments, an NSP1-like polypeptide may be expressed or over-expressed in a plant cell or organism, so as to promote the expression of at least one NIN gene(s); for example and without limitation, NLP4 and NLP9. In further embodiments, an NSP1-like polypeptide may be removed or under-expressed in a plant cell or organism, so as to decrease or eliminate the expression of at least one NIN gene(s), for example, to study the mechanism of NFB association in non-nodulating plants. In some embodiments, an NLP4 and/or NLP9 polypeptide may be expressed or over-expressed in a plant cell or organism, so as to directly increase the ability of the plant cell to associate with NFB. In further embodiments, an NLP4 and/or NLP9 polypeptide may be removed or under-expressed in a plant cell or organism, so as to decrease or eliminate the ability of the plant cell to associate with NFB. In particular embodiments, an NSP1-like, NLP4, and/or NLP9 polypeptide is expressed from a polynucleotide that is operably linked to regulatory elements that direct the expression of the polypeptide(s) in conditions other than those where nitrogen is growth limiting, thereby increasing the efficiency with which the plant utilizes environmental nitrogen under those other conditions.

In particular embodiments, the conservation of the NFB association mechanism across and between different plant species is leveraged to introduce a new NFB association phenotype into a plant via heterologous expression of a NSP1-like, NLP4, and/or NLP9 polypeptide. For example, a NSP1-like, NLP4, and/or NLP9 polypeptide may be expressed in a non-nodulating plant not normally expressing the NSP1-like, NLP4, and/or NLP9 polypeptide, so as to confer increased ability to associate with NFB upon the plant.

In alternative embodiments herein, a plant cell, plant part, and/or plant may be genetically modified to comprise at least one NSP1-like, NLP4, and/or NLP9 polynucleotide by any of several methods of introducing a heterologous molecule known in the art, thereby producing a non-natural transgenic plant cell, plant part, or plant. In particular embodiments herein, a heterologous molecule is introduced into a plant cell, plant part, and/or plant by a method selected from, for example and without limitation: transformation and selective breeding (e.g., backcross breeding).

In some embodiments, the NSP1-like, NLP4, and/or NLP9 polynucleotide is introduced such that it is operably linked to a constitutive promoter, so as to direct the expression of the gene products under conditions where they are not normally expressed (e.g., when nitrogen is not limited). In particular embodiments, the NSP1-like, NLP4, and/or NLP9 polynucleotide is introduced such that it is operably linked to a non-constitutive promoter, so as to direct the expression of the gene products in a tissue-preferred (e.g., in root tissue) or tissue-specific manner. In particular embodiments, the NSP1-like, NLP4, and/or NLP9 polynucleotide is introduced such that it is operably linked to an inducible promoter, so as to direct the expression of the gene products in a controlled manner.

Any plant species or plant cell may be genetically modified to comprise a heterologous nucleic acid herein. In some embodiments, the plant cell that is so genetically modified is capable of regeneration to produce a plant. In some embodiments, plant cells that are genetically modified (e.g., host plant cells) include cells from, for example and without limitation, a higher plant, a dicotyledonous plant, a monocotyledonous plants, a consumable plant, a crop plant, a plant utilized for its oils (e.g., an oilseed plant), and a non-nodulating plant. Such plants include, for example and without limitation: alfalfa; soybean; cotton; rapeseed (canola); linseed; corn; rice; brachiaria; wheat; safflower; sorghum; sugarbeet; sunflower; tobacco; and grasses (e.g., turf grass). In particular examples, a genetically modified plant cell or plant herein includes, for example and without limitation: Brassica napus; indian mustard (Brassica juncea); Ethiopian mustard (Brassica carinata); turnip (Brassica rapa); cabbage (Brassica oleracea); Glycine max; Linum usitatissimum; Zea mays; Carthamus tinctorius; Helianthus annuus; Nicotiana tabacum; Arabidopsis thaliana, Brazil nut (Betholettia excelsa); castor bean (Ricinus communis); coconut (Cocus nucifera); coriander (Coriandrum sativum); Gossypium spp.; groundnut (Arachis hypogaea); jojoba (Simmondsia chinensis); oil palm (Elaeis guineeis); olive (Olea eurpaea); Oryza sativa; squash (Cucurbita maxima); barley (Hordeum vulgare); sugarcane (Saccharum officinarum); Triticum spp. (including Triticum durum and Triticum aestivum); and duckweed (Lemnaceae sp.). In some embodiments, the plant may have a particular genetic background, as for elite cultivars, wild-type cultivars, and commercially distinguishable varieties.

According to methods known in the art, nucleic acids can be introduced into essentially any plant. Embodiments herein may employ any of the many methods for the transformation of plants (and production of genetically modified plants) that are known in the art. Such methods include, for example and without limitation, biological and physical transformation protocols for dicotyledenous plants, as well as monocotyledenous plants. See, e.g., Goto-Fumiyuki et al. (1999) Nat. Biotechnol. 17:282; Miki et al. (1993) Methods in Plant Molecular Biology and Biotechnology (Glick, B. R. and Thompson, J. E., Eds.), CRC Press, Inc., Boca Raton, Fla., pp. 67-88. In addition, vectors and in vitro culture methods for plant cell and tissue transformation and regeneration of plants are described, for example, in Gruber and Crosby (1993) Methods in Plant Molecular Biology and Biotechnology, supra, at pp. 89-119.

Plant transformation techniques available for introducing a nucleic acid into a plant host cell include, for example and without limitation: transformation with disarmed T-DNA using Agrobacterium tumefaciens or A. rhizogenes as the transformation agent; calcium phosphate transfection; polybrene transformation; protoplast fusion; electroporation (D'Halluin et al. (1992) Plant Cell 4:1495); ultrasonic methods (e.g., sonoporation); liposome transformation; microinjection; contact with naked DNA; contact with plasmid vectors; contact with viral vectors; biolistics (e.g., DNA particle bombardment (see, e.g., Klein et al. (1987) Nature 327:70) and microparticle bombardment (Sanford et al. (1987) Part. Sci. Technol. 5:27; Sanford (1988) Trends Biotech. 6:299, Sanford (1990) Physiol. Plant 79:206; and Klein et al. (1992) Biotechnology 10:268); silicon carbide WHISKERS-mediated transformation (Kaeppler et al. (1990) Plant Cell Rep. 9:415); nanoparticle transformation (see, e.g., U.S. Patent Publication No. US2009/0104700A1); aerosol beaming; and polyethylene glycol (PEG)-mediated uptake. In specific examples, a heterologous nucleic acid may be introduced directly into the genomic DNA of a plant cell.

A widely utilized method for introducing an expression vector into a plant is based on the natural transformation system of Agrobacterium. Horsch et al. (1985) Science 227:1229. A. tumefaciens and A. rhizogenes are plant pathogenic soil bacteria known to be useful to genetically transform plant cells. The Ti and Ri plasmids of A. tumefaciens and A. rhizogenes, respectively, carry genes responsible for genetic transformation of the plant. Kado (1991) Crit. Rev. Plant. Sci. 10:1. Details regarding Agrobacterium vector systems and methods for Agrobacterium-mediated gene transfer are also available in, for example, Gruber et al., supra, Miki et al., supra, Moloney et al. (1989) Plant Cell Reports 8:238, and U.S. Pat. Nos. 4,940,838 and 5,464,763.

If Agrobacterium is used for the transformation, the DNA to be inserted typically is cloned into special plasmids; either into an intermediate vector or a binary vector. Intermediate vectors cannot replicate themselves in Agrobacterium. The intermediate vector may be transferred into A. tumefaciens by means of a helper plasmid (conjugation). The Japan Tobacco Superbinary system is an example of such a system (reviewed by Komari et al. (2006) Methods in Molecular Biology (K. Wang, ed.) No. 343; Agrobacterium Protocols, 2^{nd} Edition, Vol. 1, Humana Press Inc., Totowa, N.J., pp. 15-41; and Komori et al. (2007) Plant Physiol. 145:1155). Binary vectors can replicate themselves both in E. coli and in Agrobacterium. Binary vectors comprise a selection marker gene and a linker or polylinker which are framed by the right and left T-DNA border regions. They can be transformed directly into Agrobacterium (Holsters, 1978). The Agrobacterium comprises a plasmid carrying a vir region. The Ti or Ri plasmid also comprises the vir region necessary for the transfer of the T-DNA. The vir region is necessary for the transfer of the T-DNA into the plant cell. Additional T-DNA may be contained.

The virulence functions of the Agrobacterium tumefaciens host will direct the insertion of a T-strand containing the construct and adjacent marker into the plant cell DNA when the cell is infected by the bacteria using a binary T DNA vector (Bevan (1984) Nuc. Acid Res. 12:8711) or the co-cultivation procedure (Horsch et al. (1985) Science 227:1229). Generally, the Agrobacterium transformation system is used to engineer dicotyledonous plants. Bevan et al. (1982) Ann. Rev. Genet 16:357; Rogers et al. (1986) Methods Enzymol. 118:627. The Agrobacterium transformation system may also be used to transform, as well as transfer, nucleic acids to monocotyledonous plants and plant cells. See U.S. Pat. No. 5,591,616; Hernalsteen et al. (1984) EMBO J 3:3039; Hooykass-Van Slogteren et al. (1984) Nature 311:763; Grimsley et al. (1987) Nature 325:1677; Boulton et al. (1989) Plant Mol. Biol. 12:31; and Gould et al. (1991) Plant Physiol. 95:426.

The genetic manipulations of a recombinant host herein may be performed using standard genetic techniques and screening, and may be carried out in any host cell that is suitable to genetic manipulation. In some embodiments, a recombinant host cell may be any organism or microorganism host suitable for genetic modification and/or recombinant gene expression. In some embodiments, a recombinant host may be a plant. Standard recombinant DNA and molecular cloning techniques used here are well-known in the art and are described in, for example and without limitation: Sambrook et al. (1989), supra; Silhavy et al. (1984) Experiments with Gene Fusions, Cold Spring Harbor Laboratory Press, Cold Spring Harbor, N.Y.; and Ausubel et al. (1987) Current Protocols in Molecular Biology, Greene Publishing Assoc. and Wiley-Interscience, New York, N.Y.

Following the introduction of a nucleic acid into a plant cell, the plant cell may be grown, and upon emergence of differentiating tissue such as shoots and roots, mature plants can be generated. In some embodiments, a plurality of plants can be generated. Methodologies for regenerating plants are known to those of ordinary skill in the art and can be found, for example, in: Plant Cell and Tissue Culture (Vasil and Thorpe, Eds.), Kluwer Academic Publishers, 1994. Genetically modified plants described herein may be cultured in a fermentation medium or grown in a suitable medium such as soil. In some embodiments, a suitable growth medium for higher plants may be any growth medium for plants, including, for example and without limitation; soil, sand, any other particulate media that support root growth (e.g., vermiculite, perlite, etc.) or hydroponic culture, as well as suitable light, water and nutritional supplements that facilitate the growth of the higher plant.

Transformed plant cells which are produced by any of the above transformation techniques can be cultured to regenerate a whole plant that possesses the transformed genotype, and thus the desired phenotype. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al. (1983) “Protoplasts Isolation and Culture,” in Handbook of Plant Cell Culture, Macmillian Publishing Company, New York, pp. 124-176; and Binding (1985) Regeneration of Plants, Plant Protoplasts, CRC Press, Boca Raton, pp. 21-73. Regeneration can also be performed from plant callus, explants, organs, pollens, embryos or parts thereof. Such regeneration techniques are described generally in Klee et al. (1987) Ann. Rev. Plant Phys. 38:467.

In other embodiments, the plant cells which are transformed are not capable of regeneration to produce a plant. Such cells may be employed, for example, in developing a plant cell line having a relevant phenotype, for example, NFB association.

A transformed plant cell, callus, tissue or plant may be identified and isolated by selecting or screening the engineered plant material for traits encoded by the marker genes present on the transforming DNA. For instance, selection can be performed by growing the engineered plant material on media containing an inhibitory amount of the antibiotic or herbicide to which the transforming gene construct confers resistance. Further, transformed plants and plant cells can also be identified by screening for the activities of any visible marker genes (e.g., the β-glucuronidase, luciferase, or gfp genes) that may be present on the recombinant nucleic acid constructs. Such selection and screening methodologies are well known to those skilled in the art.

A transgenic plant containing a heterologous molecule herein can be produced through selective breeding, for example, by sexually crossing a first parental plant comprising the molecule, and a second parental plant, thereby producing a plurality of first progeny plants. A first progeny plant may then be selected that is resistant to a selectable marker (e.g., glyphosate, resistance to which may be conferred upon the progeny plant by the heterologous molecule herein). The first progeny plant may then by selfed, thereby producing a plurality of second progeny plants. Then, a second progeny plant may be selected that is resistant to the selectable marker. These steps can further include the back-crossing of the first progeny plant or the second progeny plant to the second parental plant or a third parental plant.

It is also to be understood that two different transgenic plants can also be mated to produce offspring that contain two independently segregating, added, exogenous genes. Selfing of appropriate progeny can produce plants that are homozygous for both added, exogenous genes. Back-crossing to a parental plant and out-crossing with a non-transgenic plant are also contemplated, as is vegetative propagation. Other breeding methods commonly used for different traits and crops are known in the art. Backcross breeding has been used to transfer genes for a simply inherited, highly heritable trait into a desirable homozygous cultivar or inbred line, which is the recurrent parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting parent is expected to have the attributes of the recurrent parent (e.g., cultivar) and the desirable trait transferred from the donor parent.

A nucleic acid may also be introduced into a predetermined area of the plant genome through homologous recombination. Methods to stably integrate a polynucleotide sequence within a specific chromosomal site of a plant cell via homologous recombination have been described within the art. For instance, site specific integration as described in US Patent Publication No. 2009/0111188 A1 involves the use of recombinases or integrases to mediate the introduction of a donor polynucleotide sequence into a chromosomal target. In addition, PCT International Patent Publication No. WO 2008/021207 describes zinc finger mediated-homologous recombination to stably integrate one or more donor polynucleotide sequences within specific locations of the genome. The use of recombinases such as FLP/FRT as described in U.S. Pat. No. 6,720,475, or CRE/LOX as described in U.S. Pat. No. 5,658,772, can be utilized to stably integrate a polynucleotide sequence into a specific chromosomal site. Finally, the use of meganucleases for targeting donor polynucleotides into a specific chromosomal location is described in Puchta et al. (1996) Proc. Natl. Acad. Sci. USA 93:5055.

Other various methods for site specific integration within plant cells are generally known and applicable. Kumar et al. (2001) Trends Plant Sci. 6(4):155. Furthermore, site-specific recombination systems that have been identified in several prokaryotic and lower eukaryotic organisms may be applied for use in plants. Examples of such systems include, but are not limited too; the R/RS recombinase system from the pSRI plasmid of the yeast Zygosaccharomyces rouxii (Araki et al. (1985) J. Mol. Biol. 182:191), and the Gin/gix system of phage Mu (Maeser and Kahlmann (1991) Mol. Gen. Genet. 230:170).

Various assays can be employed in connection with the nucleic acid molecule of certain embodiments herein. In addition to phenotypic observations, the following techniques are useful in detecting the presence of a nucleic acid molecule in a plant cell. For example, the presence of the molecule can be determined by using a primer or probe of the sequence, an ELISA assay to detect an encoded protein, a Western blot to detect the protein, or a Northern or Southern blot to detect RNA or DNA. Additional techniques, such as in situ hybridization, enzyme staining, and immunostaining, also may be used to detect the presence or expression of a recombinant construct in specific plant organs and tissues.

Southern analysis is a commonly used detection method, wherein DNA is cut with restriction endonucleases and fractionated on an agarose gel to separate the DNA by molecular weight and then transferring to nylon membranes. It is then hybridized with the probe fragment which was radioactively labeled with ³²P (or other probe labels) and washed in an SDS solution.

Likewise, Northern analysis deploys a similar protocol, wherein RNA is cut with restriction endonucleases and fractionated on an agarose gel to separate the RNA by molecular weight and then transferring to nylon membranes. It is then hybridized with the probe fragment which was radioactively labeled with ³²P (or other probe labels) and washed in an SDS solution. Analysis of the RNA (e.g., mRNA) isolated from the tissues of interest can indicate relative expression levels. Typically, if the mRNA is present or the amount of mRNA has increased, it can be assumed that the corresponding transgene is being expressed. Northern analysis, or other mRNA analytical protocols, can be used to determine expression levels of an introduced transgene or native gene.

Nucleic acids herein, or segments thereof, may be used to design primers for PCR amplification. In performing PCR amplification, a certain degree of mismatch can be tolerated between primer and template. Mutations, insertions, and deletions can be produced in a given primer by methods known to an ordinarily skilled artisan.

Hydrolysis probe assay, otherwise known as TAQMAN® (Life Technologies, Foster City, Calif.), is another method of detecting and quantifying the presence of a DNA sequence. Briefly, a FRET oligonucleotide probe is designed with one oligo within the transgene and one in the flanking genomic sequence for event-specific detection. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the flanking/transgene insert sequence due to successful amplification and hybridization.

### VII. Plants, Plant Parts, and Plant Materials Comprising NSP1-Like, NLP4, and NLP9

Some embodiments herein provide plants comprising at least one heterologous NSP I-like, NLP4, and/or NLP9 polynucleotide, such as may be regenerated from stably transformed plant cells or tissues, or may be produced by introgression of such a nucleic acid from a donor line. Such plants may be used or cultivated in any manner, wherein presence of the transforming polynucleotide(s) of interest is desirable. Accordingly, transgenic plants may be engineered to, inter alia, have one or more desired traits (e.g., NFB association), by transformation, and then may be cropped and cultivated by any method known to those of skill in the art. Particular embodiments herein provide parts, cells, and/or tissues of such transgenic plants. Plant parts, without limitation, include seed, endosperm, ovule and pollen. In some embodiments, the plant part is a seed.

Representative, non-limiting example plants include non-nodulating plants; Arabidopsis; field crops (e.g. alfalfa, barley, bean, clover, corn, cotton, flax, lentils, maize, pea, rape/canola, rice, rye, safflower, sorghum, soybean, sunflower, tobacco, and wheat); vegetable crops (e.g., asparagus, beet, Brassica, broccoli, Brussels sprouts, cabbage, carrot, cauliflower, celery, cucumber (cucurbits), eggplant, lettuce, mustard, onion, pepper, potato, pumpkin, radish, spinach, squash, taro, tomato, and zucchini); fruit and nut crops (e.g., almond, apple, apricot, banana, blackberry, blueberry, cacao, cassava, cherry, citrus, coconut, cranberry, date, hazelnut, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut, and watermelon); tree woods and ornamentals (e.g., alder, ash, aspen, azalea, birch, boxwood, camellia, carnation, chrysanthemum, elm, fir, ivy, jasmine, juniper, oak, palm, poplar, pine, redwood, rhododendron, rose, and rubber).

To confirm the presence of a transforming polynucleotide(s) of interest in a regenerating plant, a variety of assays may be performed. Such assays include, for example and without limitation: biochemical assays, such as detecting the presence of a protein product, e.g., by immunological means (ELISA and/or Western blots) or by enzymatic function; plant part assays (e.g., leaf or root assays); and analysis of the phenotype of the plant.

There are numerous steps in the development of any novel, desirable plant germplasm, which may begin with the generation of a transgenic crop plant. In some embodiments, a transgenic plant comprising at least one NSP1-like, NLP4, and/or NLP9 polynucleotide may be used in a plant breeding and/or germplasm development program.

Plant breeding begins with the analysis and definition of problems and weaknesses of the current germplasm, the establishment of program goals, and the definition of specific breeding objectives. The next step is selection of germplasm that possess the traits to meet the program goals. The goal is to combine in a single variety an improved combination of desirable traits from the parental germplasm. These important traits may include higher seed yield, resistance to diseases and insects, better stems and roots, tolerance to drought and heat, and better agronomic quality.

The choice of breeding or selection methods depends on the mode of plant reproduction, the heritability of the trait(s) being improved, and the type of cultivar used commercially (e.g., F₁ hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods include pedigree selection, modified pedigree selection, mass selection, and recurrent selection.

The complexity of inheritance influences choice of the breeding method. Backcross breeding is used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.).

Promising advanced breeding lines are thoroughly tested and compared to appropriate standards in environments representative of the commercial target area(s) for three or more years. The best lines are candidates for new commercial cultivars; those still deficient in a few traits may be used as parents to produce new populations for further selection.

These processes, which lead to the final step of marketing and distribution, usually take from eight to twelve years from the time the first cross is made. Therefore, development of new cultivars is a time-consuming process that requires precise forward planning, efficient use of resources, and a minimum of changes in direction.

Pedigree breeding and recurrent selection breeding methods are used to develop cultivars from breeding populations. Breeding programs combine desirable traits from two or more cultivars or various broad-based sources into breeding pools from which cultivars are developed by selfing and selection of desired phenotypes. The new cultivars are evaluated to determine which have commercial potential.

Pedigree breeding is used commonly for the improvement of self-pollinating crops. Two parents which possess favorable, complementary traits are crossed to produce an F₁. An F₂ population is produced by selfing one or several F s. Selection of the best individuals may begin in the F₂ population; then, beginning in the F₃, the best individuals in the best families are selected. Replicated testing of families can begin in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding has been used to transfer genes for a simply inherited, highly heritable trait into a desirable homozygous cultivar or inbred line which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In embodiments herein, a NSP1-like, NLP4, and/or NLP9 polynucleotide may be introduced into a plant germplasm, for example, to develop novel inbred lines that are characterized by increased association with NFB, under the control of regulatory elements that are operably linked to the polynucleotide(s). A particular advantage of such a development program may be that the expression of an NFB phenotype, for example, in a non-nodulating plant, results in increased nitrogen utilization and/or growth.

The following examples are provided to illustrate certain particular features and/or embodiments. The examples should not be construed to limit the disclosure to the particular features or embodiments exemplified.

### EXAMPLES

### Example 1

### Materials and Methods

Arabidopsis thaliana Columbia 0 ecotype was used for all experiments unless otherwise indicated.

Growth and Treatment Conditions.

A. thaliana seedlings were grown on 0.8% agar plates with Murashige and Skoog (MS) salt media or MS salt media without nitrogen (N) supplemented with 5 mM KNO₃ as indicated, with 16:8 light:dark photoperiod (hrs.) and 22° C. constant temperature. After seven days, plants were transferred to MS salt media complete, or without N, supplemented or not with 2.5 mM NH₄NO₃.

Depending on the treatment, plants where inoculated or not with NFB or non-NFB. All bacteria were previously grown in diluted 869 medium, and 20 mL overnight culture that reached an optical density at 600 nm of 0.4 was used for inoculation. Prior to inoculation, bacteria were washed with sterile water and resuspended in 5 mL. 20 mL of plant's agar medium were inoculated with 5 mL bacteria, or alternatively non-inoculated with sterile water.

### Example 2

### Functional Association Between Non-Nodulating Plants and NFB

To determine whether A. thaliana establishes beneficial interactions for N-nutrition with NFB, we assessed the effect of different NFB species on plant growth under N-limiting conditions. We selected five different NFB species shown to fix N in association with plants:

S. meliloti RMP110 (Pichon et al. (1992) Plant Cell 4:1199),

R. etli CFN42 (Poupot et al. (1995) J. Biol. Chem. 270:6050),

C. taiwanensis LMG 19424 (Marchetti et al. (2011) Appl. Env. Microbiol. 77:2161),

B. xenovorans LB400 (Perin et al. (2006) Appl. Env. Microbiol. 72:3103), and

B. vietnamiensis G4 (Perin et al. (2006), supra).

We also tested two additional bacteria, unable to carry out BFN, as controls:

Burkholderia phytofirmans PsJN, known to enhance Arabidopsis growth (Zuniga et al. (2013) Mol. Plant Microbe Interact. 26:546), and

Cupriavidus pinatubonensis JMP134, a soil bacterium capable of associating with plants but without a positive impact on plant growth under our experimental conditions (Ledger et al. (2012) Antonie Van Leeuwenhoek 101:713).

Plants were grown on vertical plates with MS medium for seven days, and were transferred to plates with the same MS medium, but without N (MS-N), or MS-N inoculated with 104 colony forming units (cfu)/mL of the different bacteria. We evaluated dry weight of the plants seven days after transferring to MS-N.

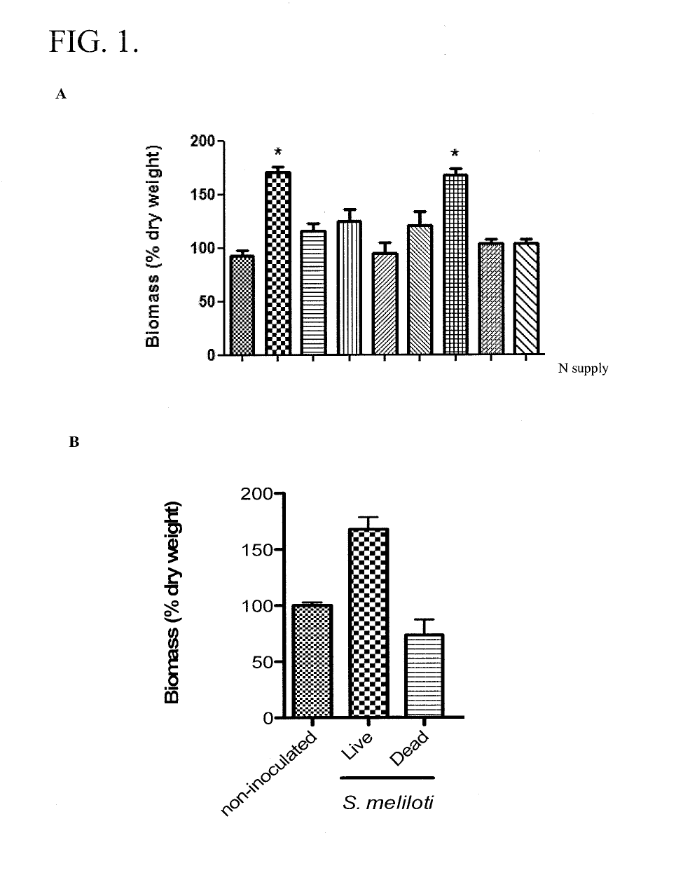

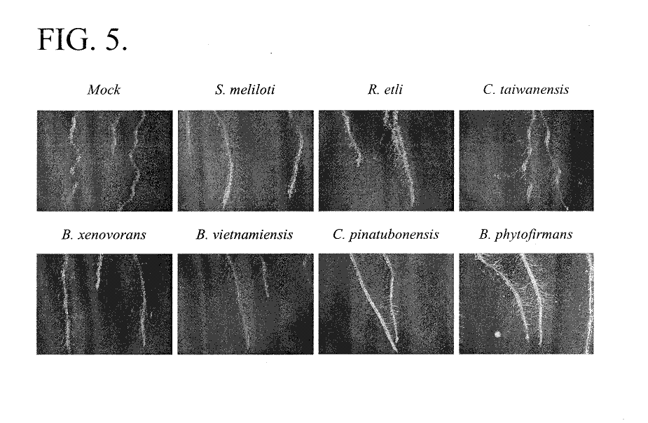

Plant dry weight was significantly higher in the presence of S. meliloti RMP110 as compared to non-inoculated medium under N-limiting conditions. FIG. 1. Moreover, plant growth was comparable to that achieved in full MS medium, under our experimental conditions. FIG. 1. These results indicate S. meliloti RMP110 can promote plant growth in the absence of an N source. None of the other bacterial species utilized were able to increase Arabidopsis dry weight, indicating mere presence of neutral or beneficial bacteria cannot explain this observation. Effective interactions between Arabidopsis and different bacteria were confirmed by a non-specific increase in root hair length observed in response to bacterial inoculation in all cases. FIG. 5.

BNF was corroborated through a ¹⁵N dilution technique, comparing plants inoculated with wild type and ΔnifH mutant under N-limiting conditions. Our results indicate that S. meliloti is able to fix atmospheric nitrogen and make it available for plant nutrition.

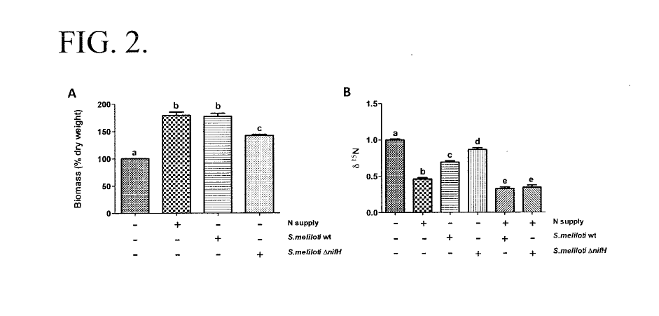

To determine whether N fixation was required for plant growth promotion under N-limiting conditions, we used a S. meliloti RMP110 mutant strain (ΔnifH) unable to fix N. Bobik et al. (2006) J. Bacteriol. 188:4890. As shown in FIG. 2A, S. meliloti RMP110 ΔnifH had a significantly reduced impact on plant growth as compared with wild-type bacteria under N-limiting conditions.

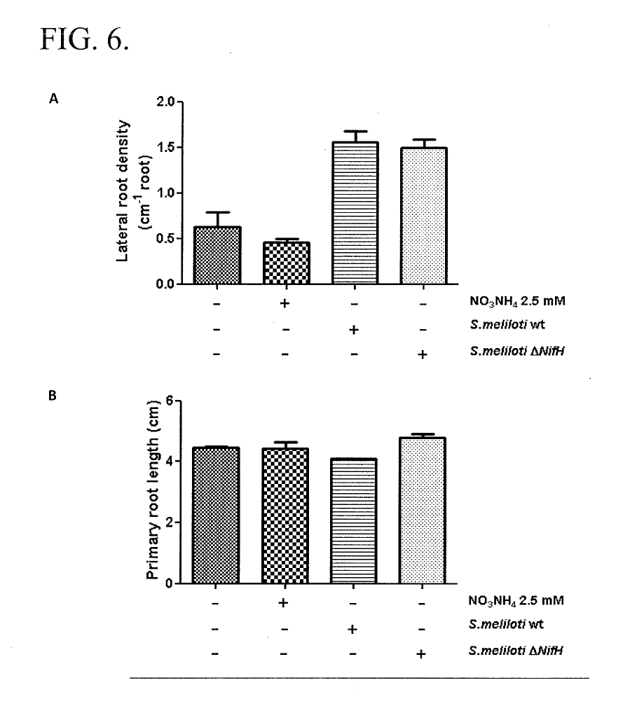

To demonstrate that growth promotion by S. meliloti RMP110 was partially due to fixed N contributing to Arabidopsis nutrition, we grew plants for seven days under MS-N supplemented with 5 mM KNO₃, which was isotopically labeled with 5% ¹⁵N. Plants were then exposed to wild-type S. meliloti RMP110 or the ΔnifH mutant in MS-N medium. Plants treated with MS-N medium supplemented with labeled KNO₃ in the presence of wild-type S. meliloti RMP110 showed reduced ¹⁵N isotopic proportion, as compared to plants inoculated with ΔnifH or non-inoculated plants. FIG. 2B. This result indicates Arabidopsis plants acquired atmospheric N, where ¹⁴N isotope predominates. Both wild-type and ΔnifH mutant bacterial strains had similar effects on lateral root density and root hair elongation, indicating both strains are able to interact with the plant. FIG. 6A.

These results show A. thaliana can functionally associate with S. meliloti RMP110 to enhance plant growth under N-limiting conditions. This plant:bacteria interaction represents an excellent model system to address non-legume plant mechanisms to promote interactions with bacterial species leading to nutritionally relevant biological N fixation

### Example 3

### Genes Regulating Arabidopsis Interaction with S. meliloti

In Arabidopsis, homologs for NSP1 and NIN genes are regulated by the presence of the bacterium only when plants are grown under N-limiting conditions. This suggests that N availability is the main factor regulating plant susceptibility to respond to the bacterium. By utilizing a functional genomics approach with plants carrying insertional mutations in NSP1-like and selected NLPs genes, we found that these transcription factors are essential for the functional association between Arabidopsis and S. meliloti.

Nitrogen fixation in legume species depends on sophisticated molecular mechanisms that control when and how symbiotic association is established with rhizobia. Oldroyd (2013) Nat. Rev. Microbiol. 11:252). In order to determine whether mechanisms may exist in Arabidopsis that regulate the interactions in our newly-discovered model system wherein Arabidopsis interacts with S. meliloti RMP110, we analyzed the function of NSP1-like and NSP2-like genes, At3g13840 (AtNSP1-like) and At4g08250 (AtNSP2-like), respectively. These genes have no known function in Arabidopsis thaliana.

Expression of AtNSP1-like and AtNSP2-like genes was evaluated in plants grown under N-sufficient conditions (5 mM KNO₃) and then transferred to 2.5 mM NH₄NO₃ or to MS-N medium in the presence or absence of S. meliloti RMP110. Plants were harvested 3 and 7 days after the transfer. Total RNA was prepared, and transcript levels for the genes of interest were measured using real-time quantitative reverse transcription polymerase chain reaction (qRT-PCR).

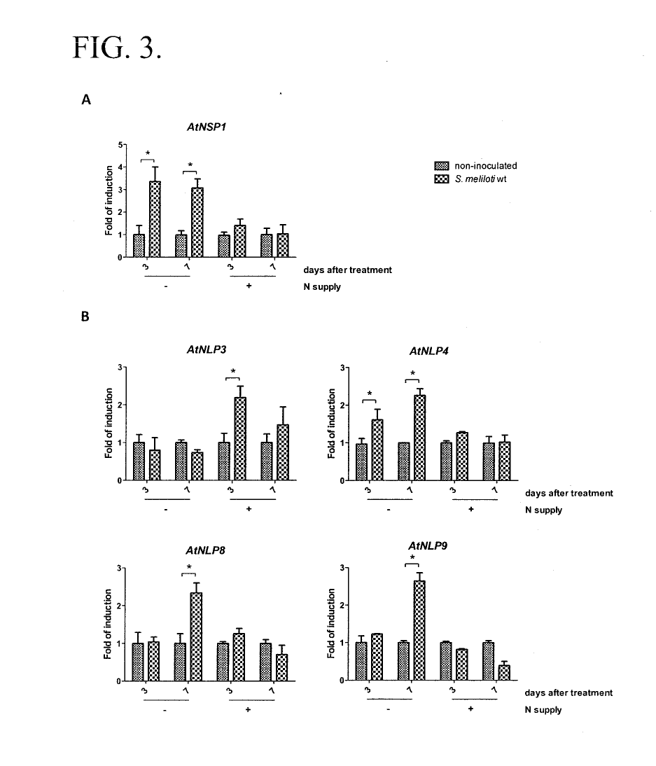

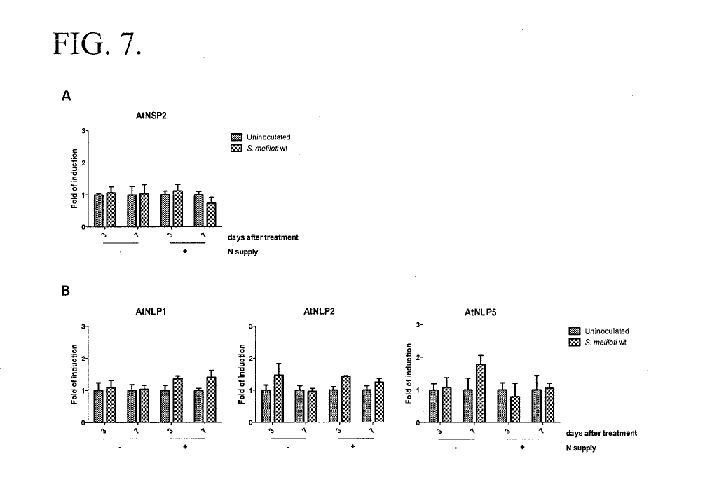

Our results showed that AtNSP1-like gene expression is induced when plants are transferred to MS-N media with bacteria, but not under other experimental conditions. FIG. 3A. This result suggests that AtNSP1-like function is required under N-limiting conditions when S. meliloti is present. In contrast, AtNSP2-like was not regulated under the experimental conditions tested. FIG. 7A.

In legumes, NSP1 and NSP2 regulate the expression of NIN genes. The Arabidopsis genome encodes nine NIN-like genes (NLPs). As a first step to evaluate the role of NLPs in the interaction of Arabidopsis and S. meliloti, we analyzed the expression of seven of these 9 NLPs (NLP1 (At2g17150); NLP2 (At4g35270); NLP3 (At4g38340); NLP4 (At1g20640); NLP5 (At1g76350); NLP8 (At2g43500); and NLP9 (At3g59580)) under the same experimental conditions described for FIG. 3A.

Our results showed that gene expression of AtNLP4, AtNLP8, and AtNLP9 is induced under N-limiting conditions and in the presence of S. meliloti RMP110 (FIG. 3B), similar to AtNSP1-like. In contrast, AtNLP3 showed a different expression pattern, being induced under N-sufficient conditions but only in the presence of bacteria. FIG. 3B. Gene expression of AtNLP1, AtNLP2, and AtNLP5 did not change significantly under the experimental conditions evaluated. FIG. 7B.

In order to address the function of AtNSP1-like, AtNLP4, AtNLP8, and AtNLP9 genes in the context of Arabidopsis and S. meliloti RMP110 interactions, two independent homozygous mutant lines of A. thaliana for AtNSP1-like (salk_{—}036071C-salk_{—}023595C), AtNLP4 (salk_{—}100786C-salk_{—}063595C), AtNLP8 (salk_{—}140298-salk_{—}031064C) and AtNLP9 (salk_{—}025839C-salk_{—}042082C) were obtained from the Arabidopsis Biological Resource Center (ABRC).

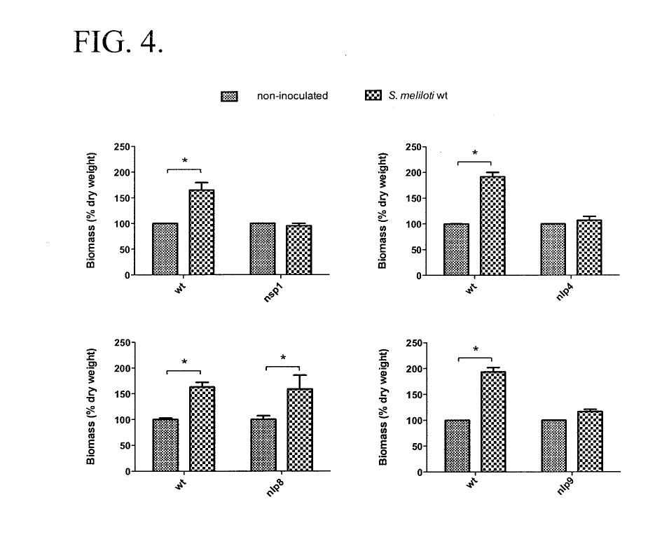

Mutant and wild-type plants grown under N-limiting conditions were inoculated with wild-type S. meliloti RMP110 (or mock-inoculated as control). Our results showed that plant growth promotion by S. meliloti RMP110 is lost in atnsp1 mutants under N-limiting conditions. FIG. 4A. Similarly, mutant plants in the AtNLP4 and AtNLP9 genes did not exhibit increased growth in the presence of S. meliloti RMP110. FIG. 4B. In contrast, atnlp8 mutant plants did not show an effect with regard to the plant growth promotion induced by S. meliloti RMP110 under N-limiting conditions. These results indicate AtNSP1-like, AtNLP4, and AtNLP9 are required for a functional interaction between Arabidopsis and S. meliloti RMP110 for enhanced growth under N-limiting conditions. These results also indicate that a conserved mechanism exists in plants to mediate plant:bacteria beneficial interactions for N nutrition that can be employed to provide BNF to non-nodulating plant species.

What may be claimed is:

1. A method for increasing nitrogen efficiency in a non-nodulating plant, the method comprising introducing at least one heterologous polypeptide into the plant to produce a transgenic plant, wherein the heterologous polypeptide is selected from the group consisting of Nodulation Signaling Pathway-like (NSPs) and NIN-like Proteins (NLPs).

2. The method according to claim 1, wherein the heterologous polypeptide is selected from the group consisting of NSP1-like, NLP4, and NLP9.

3. The method according to claim 2, wherein the heterologous polypeptide is at least 90% identical to one or more of SEQ ID NOs:1-4.

4. The method according to claim 2, wherein introducing the heterologous polypeptide into the plant comprises transforming the plant with a polynucleotide encoding the heterologous polypeptide.

5. The method according to claim 4, wherein the polynucleotide is at least 80% identical to one or more of SEQ ID NOs:4-6.

6. The method according to claim 4, wherein the polynucleotide hybridizes under stringent conditions to one or more of SEQ ID NOs:4-6.

7. The method according to claim 4, wherein the polynucleotide is codon-optimized for expression of the heterologous polypeptide in the plant.

8. The method according to claim 4, wherein the polynucleotide is operably linked to a plant promoter selected from the group consisting of constitutive promoters, tissue-preferred promoters, tissue-specific promoters, and inducible promoters.

9. The method according to claim 1, wherein the increased nitrogen efficiency comprises increased growth of the transgenic plant under limited nitrogen conditions.

10. The method according to claim 1, wherein the heterologous polypeptide is introduced into root tissue of the plant.

11. The method according to claim 1, wherein the method comprises growing the transgenic plant in limited nitrogen conditions.

12. A transgenic plant produced by the method according to claim 1.

13. A cell, tissue, seed, or material obtained from the transgenic plant of claim 12.

14. A nucleic acid molecule comprising a polynucleotide having an agronomic function, wherein the polynucleotide is selected from the group consisting of a polynucleotide that is at least 80% identical to SEQ ID NO:4; a polynucleotide that hybridizes under stringent conditions to a nucleic acid consisting of SEQ ID NO:4; a polynucleotide that is at least 80% identical to SEQ ID NO:5; a polynucleotide that hybridizes under stringent conditions to a nucleic acid consisting of SEQ ID NO:5; a polynucleotide that is at least 80% identical to SEQ ID NO:6; and a polynucleotide that hybridizes under stringent conditions to a nucleic acid consisting of SEQ ID NO:6; wherein said nucleic acid sequence is operably linked to a heterologous promoter.

15. A transgenic plant cell stably transformed with the nucleic acid molecule of claim 14.

16. A method for producing a transgenic plant, the method comprising introducing a nucleic acid into a cell of the plant, wherein the nucleic acid comprises a nucleotide sequence encoding a heterologous Nodulation Signaling Pathway 1-like (NSP1-like), NIN-like Protein-4 (NLP4), or NIN-like Protein-9 (NLP9) polypeptide, thereby producing a transgenic plant.

17. The method according to claim 16, wherein the plant is a non-nodulating plant.

18. A transgenic plant produced by the method according to claim 16.

19. A cell, tissue, seed, or material obtained from the transgenic plant of claim 17.

20. The transgenic plant of claim 17, wherein the transgenic plant comprises increased growth of the transgenic plant under limited nitrogen conditions, as compared to a wild-type plant of the same species.

21. A method for increasing nitrogen efficiency in a non-nodulating plant, the method comprising introducing into the plant at least one means for promoting association with nitrogen-fixing bacteria (NFB), wherein the plant's nitrogen efficiency is increased through biological nitrogen fixation (BNF).

22. The method according to claim 21, wherein the means for promoting association with nitrogen-fixing bacteria NFB is selected from the group consisting of a polypeptide consisting of one of SEQ ID NOs:1, 3, and 4, and a polynucleotide consisting of one of SEQ ID NOs:4-6.


SEQUENCE LISTING (6 sequences)

SEQ ID NO: 1 - protein, 510 aa, Arabidopsis thaliana
MLLEETEPPNQTLDHVLSWLEDSVSLSPLPGFDDSYLLHEFDGSQTWEWDQTQDPEHGFIQSYSQDLSAAYVGCEATNLEVVTEAPSIDLDLPPEIQQPNDQSRKRSHDGFLEAQQVKKSARSKRKAIKSSEKSSKDGNKEGRWAEKLLNPCALAITASNSSRVQHYLCVLSELASSSGDANRRLAAFGLRALQHHLSSSSVSSSFWPVFTFASAEVKMFQKTLLKFYEVSPWFALPNNMANSAILQILAQDPKDKKDLHIIDIGVSHGMQWPTLLEALSCRLEGPPPRVRITVISDLTADIPFSVGPPGYNYGSQLLGFARSLKINLQISVLDKLQLIDTSPHENLIVCAQFRLHHLKHSINDERGETLKAVRSLRPKGVVLCENNGECSSSADFAAGFSKKLEYVWKFLDSTSSGFKEENSEERKLMEGEATKVLMNAGDMNEGKEKWYERMREAGFFVEAFEEDAVDGAKSLLRKYDNNWEIRMEDGDTFAGLMWKGEAVSFCSLWK

SEQ ID NO: 2 - protein, 844 aa, Arabidopsis thaliana
MEDSFLQSENVVMDADFMDGLLLDGCWLETTDGSEFLNIAPSTSSVSPFDPTSFMWSPTQDTSALCTSGVVSQMYGQDCVERSSLDEFQWNKRWWIGPGGGGSSVTERLVQAVEHIKDYTTARGSLIQLWVPVNRGGKRVLTTKEQPFSHDPLCQRLANYREISVNYHFSAEQDDSKALAGLPGRVFLGKLPEWTPDVRFFKSEEYPRVHHAQDCDVRGTLAIPVFEQGSKICLGVIEVVMTTEMVKLRPELESICRALQAVDLRSTELPIPPSLKGCDLSYKAALPEIRNLLRCACETHKLPLAQTWVSCQQQNKSGCRHNDENYIHCVSTIDDACYVGDPTVREFHEACSEHHLLKGQGVAGQAFLTNGPCFSSDVSNYKKSEYPLSHHANMYGLHGAVAIRLRCIHTGSADFVLEFFLPKDCDDLEEQRKMLNALSTIMAHVPRSLRTVTDKELEEESEVIEREEIVTPKIENASELHGNSPWNASLEEIQRSNNTSNPQNLGLVFDGGDKPNDGFGLKRGFDYTMDSNVNESSTFSSGGFSMMAEKKRTKADKTITLDVLRQYFAGSLKDAAKNIGVCPTTLKRICRQHGIQRWPSRKIKKVGHSLQKIQRVIDSVQGVSGPLPIGSFYANFPNLVSQSQEPSQQAKTTPPPPPPVQLAKSPVSSYSHSSNSSQCCSSETQLNSGATTDPPSTDVGGALKKTSSEIELQSSSLDETILTLSSLENIPQGTNLLSSQDDDFLRIKVSYGEEKIRLRMRNSRRLRDLLWEIGKRFSIEDMSRYDLKYLDEDNEWVLLTCDEDVEECVDVCRTTPSHTIKLLLQASSHHFPERSSATEYSLWH

SEQ ID NO: 3 - protein, 894 aa, Arabidopsis thaliana
MENPSASRDNKGFCFPDIPVEEMDGWVKNLISEEDMFSSSSTSELMNFESFASWCNSPSAADILFTQYGLSTSQSIIPFGGLEGSYACEKRPLDCTSVPRSLSHSLDEKMLKALSLFMEFSGEGILAQFWTPIKTGDQYMLSTCDQAYLLDSRLSGYREASRRFTFSAEANQCSYPGLPGRVFISGVPEWTSNVMYYKTAEYLRMKHALDNEVRGSIAIPVLEASGSSCCAVLELVTCREKPNFDVEMNSVCRALQAVNLQTSTIPRRQYLSSNQKEALAEIRDVLRAVCYAHRLPLALAWIPCSYSKGANDELVKVYGKNSKECSLLCIEETSCYVNDMEMEGFVNACLEHYLREGQGIVGKALISNKPSFSSDVKTFDICEYPLVQHARKFGLNAAVATKLRSTFTGDNDYILEFFLPVSMKGSSEQQLLLDSLSGTMQRLCRTLKTVSDAESIDGTEFGSRSVEMTNLPQATVSVGSFHTTFLDTDVNSTRSTFSNISSNKRNEMAGSQGTLQQEISGARRLEKKKSSTEKNVSLNVLQQYFSGSLKDAAKSLGVCPTTLKRICRQHGIMRWPSRKINKVNRSLRKIQTVLDSVQGVEGGLKFDSVTGEFVAVGPFIQEFGTQKSLSSHDEDALARSQGDMDEDVSVEPLEVKSHDGGGVKLEEDVETNHQAGPGSLKKPWTWISKQSGLIYSDDTDIGKRSEEVNKDKEDLCVRRCLSSVALAGDGMNTRIERGNGTVEPNHSISSSMSDSSNSSGAVLLGSSSASLEQNWNQIRTHNNSGESGSSSTLTVKATYREDTVRFKLDPYVVGCSQLYREVAKRFKLQEGAFQLKYLDDEEEWVMLVTDSDLHECFEILNGMRKHTVKFLVRDIPNTAMGSSAGSNGYLGTGT

SEQ ID NO: 4 - DNA, 1533 bp, Arabidopsis thaliana
atgttgttggaagaaacagaaccaccaaaccagactctagatcatgtcctaagctggcttgaggattctgtgtccttatccccattaccaggatttgatgattcttatttgctccacgagtttgatgggtctcaaacgtgggaatgggatcagactcaagatccggagcatggttttattcaaagctatagtcaagatcttagtgcagcatatgttggttgtgaagcaactaacctggaagtggtaacagaagctccatccattgatttggatcttccacctgaaattcagcaaccaaacgatcagtccaggaaaaggagccacgacgggtttctcgaggcacaacaggtgaaaaaatcggcaaggagcaagagaaaagcaatcaagtctagtgagaagagctccaaagatggtaacaaggaagggagatgggcagagaagttgcttaacccttgtgccttggccattacggcaagtaactcatcaagggttcaacattacctttgtgttctctctgaactggcctcttcttctggtgatgctaatcgtcggcttgcagcttttggtcttcgggctttgcaacatcatctttcctcatcctctgtgtcatcatccttttggcctgtttttacttttgcttcagcggaagtgaagatgtttcaaaagactctgcttaagttctacgaggtaagcccttggtttgctttgcctaacaacatggcaaactcagctatcctgcagattttagcacaggatcccaaagataaaaaggatcttcatattattgatattggtgtttctcacggtatgcaatggcccactttgttggaggctctgagctgcagactagaaggacctcctcctcgtgttcgaataaccgttatatcagatctaaccgcagacatacctttctctgttggtccaccaggttacaattatggttctcaactcctaggctttgctcggtctctcaagatcaaccttcagattagtgtacttgacaagttacaactcattgatacctcacctcacgagaacttaatcgtgtgtgctcagtttaggctgcatcacctgaagcatagcatcaatgatgagagaggcgagactttgaaagcagtgagaagtttaaggccaaaaggagtggttctttgtgagaacaatggagaatgcagtagtagtgcggactttgcagcaggattctcgaagaaactggagtatgtatggaagtttctggattcaacaagctcgggatttaaagaagagaatagcgaagagagaaaactaatggaaggagaggcaacaaaggtgttgatgaatgcaggagatatgaatgaaggaaaagagaaatggtatgagaggatgagagaagctggtttttttgtagaagcatttgaagaagatgcagttgatggagccaaatccttactaagaaagtatgacaacaattgggaaataagaatggaagatggagatacctttgctggattaatgtggaaaggagaggcagtttccttttgttcattgtggaagtag

SEQ ID NO: 5 - DNA, 2535 bp, Arabidopsis thaliana
atggaagatagtttccttcaatctgagaacgtggttatggacgctgacttcatggatggattgttactagatggttgttggttagagactacagatggatctgagtttcttaacatagctccttcaacttcttctgttagcccttttgatccaacttccttcatgtggtctccaactcaagatacatcagctctttgcacatcaggagttgtatctcagatgtatggtcaggattgtgtagaaagatctagtcttgatgagtttcaatggaacaaacgatggtggattggaccaggaggtggtggttcttcggttactgagaggttggttcaagcagttgaacacattaaagattacacaacagcgagaggctcacttattcagttatgggttccggttaatagaggcggtaagcgagttttgaccacaaaggaacaaccttttagccatgatccgttgtgtcaaagacttgcaaactatagagagatctctgtgaattatcacttctctgctgagcaagatgattccaaggctttagctggtttgcctgggagggttttcttggggaagcttcctgaatggactcctgatgttaggtttttcaagagcgaggagtatccgagagtacaccatgctcaggactgcgatgtccgtggaacgctggcgattccggtgtttgaacaaggtagtaagatttgcttgggtgttattgaggttgtaatgaccactgagatggttaaactaagacctgagcttgaaagcatttgcagagcacttcaggcagttgatcttaggagcaccgagcttccgattccaccttctctaaagggatgtgacttatcctacaaagctgccttacctgaaatccgaaacctcttgagatgtgcttgtgagactcataaactacctttagctcagacatgggtttcttgtcaacagcaaaacaaaagcgggtgccgtcacaacgatgagaactacatccattgcgtatcaaccattgatgatgcttgctacgttggtgatccaacagttcgtgagttccatgaagcttgctctgagcatcacctcttgaaaggccaaggagttgcaggtcaagccttcttgaccaatggaccttgcttttcatctgatgtatctaactacaagaaatcagagtaccctctctctcaccatgctaatatgtacggtttacatggcgcggttgcaattcgcctgcggtgcatccacacgggctctgctgatttcgtcttagagttctttttgcctaaagactgcgatgatctggaggaacagaggaaaatgttgaatgctctttcaactattatggctcatgtgcctagaagcttaaggactgttacagacaaagaactagaagaagagagtgaagtgatagagagggaagagatagtaacgccaaagatagaaaacgcatctgaactccacggaaattccccatggaatgcctctcttgaagaaatccagcggagtaataatactagtaatcctcagaatcttggactggtatttgatggaggagacaaaccaaatgatggttttggcttaaaaagaggttttgactacaccatggattctaatgtcaatgagagcagcactttctctagtggtggtttcagtatgatggccgagaaaaagcgtacaaaagcagataaaaccatcactttggatgttcttcgacagtatttcgctgggagcttgaaagatgcagccaagaatatcggtgtttgtccaacgaccttgaagagaatatgcagacagcatggtatacaaagatggccttcaagaaagataaaaaaagtgggacattctctgcagaagatccaacgagtgattgattcggttcaaggtgtttctggtcctcttcccataggctcattctatgcaaatttccccaatttagtctcacagtcacaagaaccatcacaacaagccaagaccacgcctcctcctccgccgccagtgcagcttgcaaagtcccctgtatcctcgtatagtcacagttcaaactctagccaatgttgctccagtgaaacccaactaaacagcggtgcaacaaccgatcctccttcaactgatgtaggaggtgcattgaagaagacgagcagcgaaatcgagcttcaaagctcgagtcttgacgagacaattttgactctctccagtttagaaaacatccctcaaggcaccaacttgttatcatctcaagatgatgactttctgaggattaaagttagctacggagaagagaagatcagattacggatgcggaattcgcgcaggttaagagatctattgtgggagattgggaagcggtttagcatagaggatatgagcaggtatgatctaaagtacttagacgaagacaatgaatgggttttgttgacttgcgacgaagatgtagaagagtgtgtagatgtctgcagaactacaccgagtcataccattaagcttttgcttcaggcttcttctcatcatttccctgaacgttcttcagctactgaatacagtttatggcactga

SEQ ID NO: 6 - DNA, 2685 bp, Arabidopsis thaliana
atggagaacccatcagcatccagagataataaaggtttctgttttccagatattccagtagaagaaatggatggctgggttaagaatttgatctctgaagaagatatgtttagctcctcttcaacttcagagcttatgaatttcgaatcttttgcttcatggtgcaacagcccttccgctgcagatatcttgttcactcaatacggtttatcgacctctcaatctattatacctttcggaggcttagaaggctcatacgcttgcgagaaaagaccgttagactgtactagtgttccaaggtcattgagccattctcttgatgagaagatgctcaaagcattaagtttgtttatggagttctctggagagggaattctggcacagttttggactcctattaagacaggagatcagtacatgcttagtacttgtgatcaggcgtatctgcttgactcgaggctatctggataccgtgaagcgtcgaggagattcactttctctgctgaagcaaatcaatgctcttatccaggtcttccaggcagagtctttatctctggagttcctgagtggacatcaaacgttatgtattacaagactgctgaatatttaaggatgaagcatgcattagataacgaagtccgtggttcgattgcaattcctgtccttgaagcatcaggttcttcttgttgtgcagttctggaacttgtgacatgtagggaaaaaccaaactttgatgtggagatgaactctgtttgccgtgctctgcaggccgtgaacttacaaacatcaactattcctcgtcgccagtacctttcaagtaatcaaaaagaagctttggctgaaataagagatgttctcagagcagtgtgctatgcacataggttgcctttagctctagcttggattccctgtagttactccaaaggagcaaacgatgagttggtaaaggtttatggaaaaaactcaaaggaatgttctcttctttgcatagaagagacatcatgttatgtgaatgatatggaaatggaaggctttgtgaatgcatgtttggagcattatctaagagaagggcaaggaattgttggcaaagcactcatatcaaacaaaccgtctttctcatctgatgtaaagacatttgatatctgcgagtaccctcttgttcaacatgctcgaaagtttggtcttaatgctgcagttgctaccaaactgaggagcacattcactggtgacaatgactatatacttgagttttttttacctgtaagtatgaagggaagctcagaacaacaacttttgctggacagtctctcgggcaccatgcagagactatgtcggactctgaaaactgtttcagatgctgaatcaattgacggtacagaatttggatctcgtagtgtagaaatgacaaatctcccacaggctactgtatccgttggaagctttcatacgacatttcttgatactgacgtcaactctactcgaagtaccttttcgaacatctcctctaataaaagaaatgaaatggcaggttctcaaggcactcttcagcaggaaattagcggagcaagaagattagagaagaagaaaagcagtacagagaagaatgtgagcttaaatgttctccaacaatacttctctgggagcttaaaggatgctgcaaaaagccttggtgtttgtccgactacactaaaaaggatatgtagacaacacggaattatgagatggccatctcgaaagattaacaaagtgaataggtcactaaggaaaatacagacggtgcttgactctgtccagggtgtagaaggaggactgaagtttgactcggtgacaggggaatttgtagcagttggcccttttatacaagaatttgggacccaaaagagtctgtcttctcatgatgaagatgcacttgcaagaagccaaggtgatatggatgaagatgtgtcagtagagcctttggaagttaaatctcatgatggtggcggtgtcaagttggaggaggatgttgaaacaaaccaccaagcgggaccaggatccttgaagaagccatggacttggataagcaaacagtctggcttgatctatagtgatgataccgacataggaaaaagaagtgaagaggtaaacaaggataaagaagacctttgtgttcgaaggtgcttgagctctgtagcacttgcaggtgatggaatgaatacaagaatcgagcgaggtaatggaactgtagaaccaaaccactccatatcaagtagcatgtcggattcatcaaatagctcaggagcagttttgctgggaagttcatctgcttccttggaacaaaactggaaccaaataagaactcataacaatagcggtgaaagcggatcaagttcaacactaaccgtaaaagccacttacagagaggacactgtacgtttcaagcttgatccatacgttgttgggtgttctcagctctacagagaagtggctaagcgtttcaagctgcaagaaggtgcctttcagttgaaatacttggatgatgaagaagaatgggtgatgttggtcacagattctgatctccatgaatgcttcgagatattaaatggtatgagaaaacatacagtgaagtttctggtccgtgatataccgaacaccgcaatgggaagttccgcaggcagcaatggttacctcggaacaggcacctaa